(19) United States
(12) Patent Application Publication
(10) Pub. No.: US 2024/0410887 A1
(43) Pub. Date: Dec. 12, 2024

(54) USE OF BIOMARKERS IN DIAGNOSING AND TREATING LUPUS NEPHRITIS

(71) Applicant: The Johns Hopkins University, Baltimore, MD (US)

(72) Inventors: Andrea Fava, Baltimore, MD (US); Michelle Petri, Baltimore, MD (US)

(21) Appl. No.: 18/703,915

(22) PCT Filed: Oct. 31, 2022
(86) PCT No.: PCT/US2022/048344

§ 371 Date: Apr. 23, 2024

Related U.S. Application Data
(60) Provisional application No. 63/276,359, filed on Nov. 5, 2021.

(51) Int. Cl. G01N 33/564; G01N 33/68

(52) U.S. Cl. CPC G01N 33/564; G01N 33/6863; G01N 2800/34

(57) ABSTRACT

The present disclosure relates to methods of diagnosing lupus nephritis or proliferative lupus nephritis in a subject, methods of predicting a subject's risk of developing lupus nephritis and methods of determine whether a subject suffering from lupus nephritis is responding to treatment. The methods herein involve detecting the presence of or level of at least one biomarker which is IL-16, Galectin-1, CD163, CD206, FOLR2, proteinase 3, or a combination thereof.

### RELATED APPLICATION INFORMATION

This application claims priority to U.S. provisional patent application Ser. No. 63/276,359, filed Nov. 5, 2021, which is incorporated herein by reference in its entirety.

### STATEMENT OF GOVERNMENTAL INTEREST

This invention was made with government support under grant AR067679 and AR069572 awarded by the National Institutes of Health. The government has certain rights in the invention.

### BACKGROUND

Lupus nephritis (LN) is a severe manifestation of systemic lupus erythematosus (SLE) that frequently leads to end-stage kidney disease despite treatment (1, 2). Diagnosis, classification, and treatment of LN rely on histopathological features of kidney biopsies in patients with proteinuria. Kidney biopsies are necessary because proteinuria neither distinguishes treatable inflammation from chronic damage nor differentiates LN classes. Furthermore, proteinuria does not correlate with intrarenal inflammation, and it is a lagging indicator as it occurs after damage has occurred. Kidney biopsies have an indispensable role in that they can distinguish active nephritis from chronic damage, both of which manifest with proteinuria. However, kidney biopsies have also limitations. Most notably, histology does not capture patient-specific active biological pathways. Further, the histological class frequently changes on repeat kidney biopsies, suggesting that the histological classification may artificially divide patients based on one point in time (3, 4). Procedure-related complications may occur (5), and up to 35% of kidney biopsies may fail to obtain an adequate sample (6). Access to kidney biopsies may delay diagnosis and treatment, and can be limited by antithrombotic and anticoagulation treatments, severe thrombocytopenia, and in resource poor settings. Finally, because the presence of proteinuria implies that underlying kidney damage has already happened, kidney biopsies are a lagging indicator. Thus, there is a pressing need for a noninvasive biomarker to probe in “real-time” the active molecular pathological processes in the kidney and to monitor them over time in response to treatment.

Several available biomarkers correlate with histological features, but none are currently used in clinical practice (7, 8). These lack the sensitivity and specificity to detect active renal inflammation, predict flares, and reliably inform prognosis, and do not add actionable information in addition proteinuria or renal function (7, 8). Unbiased proteomic screenings carry a high potential for discovery, but these have been limited to the evaluation of proteins or peptides sufficiently abundant to be detectable by mass spectrometry (9, 10).

More sensitive aptamer-based arrays identified candidate urinary biomarkers associated with proteinuria, but their ability to predict nephritis activity and clinical outcomes is still to be determined (11).

Management of LN could be greatly enhanced by a resource which can identify candidate biomarkers that predict histological features and clinical outcomes, as well as infer the renally active biologically pathways.

### SUMMARY

In one embodiment, the present disclosure relates to a method of diagnosing lupus nephritis, such as, proliferative lupus nephritis, in a subject. The method comprises the steps of:
(a) obtaining a biological sample from a subject;
(b) detecting a presence of at least one biomarker in the sample, wherein at least one of the biomarkers is IL-16, Galectin-1, CD163, CD206, FOLR2, proteinase 3 (PRTN3), or a combination thereof and optionally, at least one biomarker in Table 1; and
(c) diagnosing the subject as having lupus nephritis, such as proliferative lupus nephritis, if at least one of IL-16, Galectin-1, CD163, CD206, FOLR2, or PRTN3 and optionally, at least one biomarker in Table 1 is detected in the sample.

In some aspects, the sample in the above method can be whole blood, serum, plasma, or urine. In some aspects, the subject is a human who may or may not have systemic lupus erythematosus. In other aspects, the subject is a human who has systemic lupus erythematosus but may or may not have proteinuria.

In some aspects of the above method, the biomarkers being detected are at least two of IL-16, Galectin-1, CD163, CD206, FOLR2, and PRTN3 and optionally, at least one biomarker in Table 1. In other aspects, the biomarkers being detected are at least three of IL-16, Galectin-1, CD163, CD206, FOLR2, and PRTN3 and optionally, at least one biomarker in Table 1. In yet other aspects, the biomarkers being detected are each of IL-16, Galectin-1, CD163, CD206, FOLR2, and PRTN3 and optionally, at least one biomarker in Table 1. In other aspects, the presence of at least one of these biomarkers in the sample is displayed on an instrument.

In other aspects, the above method further comprises treating the subject diagnosed with lupus nephritis, such as proliferative lupus nephritis, with at least one immunosuppressant, at least one corticosteroid, at least one B-lymphocyte stimulator specific inhibitor, rituximab, or any combination thereof.

In another embodiment, the present disclosure relates to a method of predicting a subject's risk of developing lupus nephritis, such as proliferative lupus nephritis. In this aspect, the method comprises the steps of:
(a) obtaining a biological sample from a subject;
(b) determining a level at least one biomarker in the sample, wherein at least one of the biomarkers is IL-16, Galectin-1, CD163, CD206, FOLR2, proteinase 3 (PRTN3), or a combination thereof and optionally, at least one biomarker in Table 1;
(c) comparing the level of the at least one biomarker determined in step (b) with a control level for the same biomarker; and
(d) predicting whether the subject at risk of developing lupus nephritis, such as proliferative lupus nephritis, based on the comparison in step (c).

In some aspects, the sample in the above method can be whole blood, serum, plasma, or urine. In some aspects, the subject is a human who may or may not have systemic lupus erythematosus. In other aspects, the subject is a human who has systemic lupus erythematosus but may or may not have proteinuria.

In some aspects, in the above method further comprises determining that the subject is at risk of developing lupus nephritis, such as, proliferative lupus nephritis, when the level of the at least one biomarker determined in step (b) is higher than the control level. In other aspects, the above method further comprises determining that the subject is not at risk of developing lupus nephritis, such as, proliferative lupus nephritis, when the level of the at least one biomarker determined in step (b) is lower than the control level.

In some aspects of the above method, the biomarkers being detected are at least two of IL-16, Galectin-1, CD163, CD206, FOLR2, and PRTN3 and optionally, at least one biomarker in Table 1. In other aspects, the biomarkers being detected are at least three of IL-16, Galectin-1, CD163, CD206, FOLR2, and PRTN3 and optionally, at least one biomarker in Table 1. In yet other aspects, the biomarkers being detected are each of IL-16, Galectin-1, CD163, CD206, FOLR2, and PRTN3 and optionally, at least one biomarker in Table 1. In other aspects, the presence of at least one of these biomarkers in the sample is displayed on an instrument.

In the above method, if the subject is determined to be at risk of developing lupus nephritis, such as, proliferative lupus nephritis, then the method further comprises treating the patient for lupus nephritis, such as proliferative lupus nephritis, if the subject is determined to be at risk of developing lupus nephritis, such as, proliferative lupus nephritis. In such an aspect, the subject can be treated with at least one immunosuppressant, at least on corticosteroid, a B-lymphocyte stimulator specific inhibitor, rituximab, or a combination thereof. For example, the at least one immunosuppressant can be cyclosporine, tacrolimus, a calcineurin-inhibitor, cyclophosphamide, hydroxychloroquine, azathioprine, mycophenolate, or any combination thereof. The corticosteroid can be prednisone, prednisolone, methylprednisolone, or any combination thereof.

In yet another embodiment, the present disclosure relates to a method of determining whether a subject suffering from lupus nephritis, such as, proliferative lupus nephritis, is responding to treatment for the lupus nephritis. In this aspect, the method comprises the steps of:
(a) obtaining a biological sample from a subject being treated for lupus nephritis, such as, proliferative lupus nephritis;
(b) determining a level at least one biomarker in the sample, wherein at least one of the biomarkers are IL-16, Galectin-1, CD163, CD206, FOLR2, proteinase 3 (PRTN3), or a combination thereof and optionally, at least one biomarker in Table 1;
(c) comparing the level of the at least one biomarker determined in step (b) with a control level for the same biomarker; and
(d) determining that the subject is responding to treatment for lupus nephritis if the level of the at least one biomarker determined in step (b) is less than the control level for the same biomarker or that the subject is not responding to treatment for lupus nephritis, such as, proliferative lupus nephritis, if the level of the at least one biomarker determine in step (b) is the same as or greater than the control level for the same biomarker.

In some aspects of the above method, the control level for the biomarker being determined is the level of the biomarker obtained from the subject prior to the subject beginning or starting treatment for lupus nephritis, such as proliferative lupus nephritis.

In some aspects, the sample in the above method can be whole blood, serum, plasma, or urine. In some aspects, the sample in the above method is obtained from the subject at 3 months after kidney treatment. In some aspects, the subject is a human who has systemic lupus erythematosus but may or may not have proteinuria.

In some aspects of the above method, the biomarkers being detected are at least two of IL-16, Galectin-1, CD163, CD206, FOLR2, and PRTN3 and optionally, at least one biomarker in Table 1. In other aspects, the biomarkers being detected are at least three of IL-16, Galectin-1, CD163, CD206, FOLR2, and PRTN3 and optionally, at least one biomarker in Table 1. In yet other aspects, the biomarkers being detected are each of IL-16, Galectin-1, CD163, CD206, FOLR2, and PRTN3 and optionally, at least one biomarker in Table 1. In other aspects, the presence of at least one of these biomarkers in the sample is displayed on an instrument.

In some aspects of the above method, the method comprises monitoring the subject receiving treatment for lupus nephritis, such as, proliferative lupus nephritis.

In some aspects, the subject is being treated with at least one immunosuppressant, at least on corticosteroid, a B-lymphocyte stimulator specific inhibitor, rituximab, or a combination thereof. For example, the at least one immunosuppressant can be cyclosporine, tacrolimus, a calcineurin-inhibitor, cyclophosphamide, hydroxychloroquine, azathioprine, mycophenolate, or any combination thereof. The corticosteroid can be prednisone, prednisolone, methylprednisolone, or any combination thereof.

In still yet another embodiment, the present disclosure relates to a method for determining a type or grade of lupus nephritis in a subject, the method comprising the steps of:
(a) obtaining a biological sample from a subject being treated for lupus nephritis;
(b) determining a level at least one biomarker in the sample, wherein at least one of the biomarkers are IL-16, CD163, Catalase, PRTN3, S100A8, Azurocidin, and MMP8, or a combination thereof; and
(c) determining that the type or grade of the lupus nephritis is proliferative when the level of the at least one biomarker determined in step (b) is higher than the control level for the same biomarker or that the type or grade of the lupus nephritis is pure membranous when the level of the at least one biomarker determined in step (b) is less than the control level for the same biomarker.

In another aspect, the present disclosure relates to an article of manufacture which comprises a set of reagents to measure the levels of a panel of biomarkers in a biological sample, wherein the panel of biomarkers comprises IL-16, Galectin-1, CD163, CD206, FOLR2, and proteinase 3 (PRTN3) and optionally, at least one biomarker in Table 1, and where the set of reagents are bound to a solid support and specifically binds to the biomarkers. In some aspects, the reagents in the article of manufacture are specific binding molecules or agents. For example, such reagents can be an antibody or antigen-binding fragment thereof, a peptide or a fragment thereof, or a combination thereof. In some aspects, the solid support in the article of manufacture is a biochip, a microtiter plate, a stick, a bead, or any combination thereof.

In yet another embodiment, the present disclosure relates to a test kit comprising the above-described set of reagents.

### BRIEF DESCRIPTION OF THE DRAWINGS

FIG. 1 shows the identification of pathogenic pathways by urine proteomics. (A) Volcano plot illustrating the differential abundance of 1000 urinary proteins in lupus nephritis (LN, n=30) and healthy controls (n=7). There were 237 proteins significantly more abundant in LN (>2 fold, FDR <10%, moderated t test). (B) Heatmap of the abundance of the 12 non-overlapping pathways enriched in LN urine samples by pathway enrichment analysis (GO Biological Process). Twenty of the thirty patients displayed a LN cluster with higher abundance of all pathways, whereas the patients in the other cluster exhibited an intermediate abundance as compared to healthy controls. Clustering was otherwise not explained by other clinical variables such as proteinuria, renal function, nephritis activity, chronic damage, or class. Values were scaled by rows. Clustering was performed using the Ward's minimum variance method.

FIG. 2 shows the proteomic profile of proliferative lupus nephritis. (A) Volcano plot illustrating the differential abundance of 1000 urinary proteins in proliferative LN (n=14) and pure membranous LN (n=9). (B) Pathway enrichment analysis (GO Biological Process) of the urinary proteomic profile revealed that chemotaxis was the process most enriched in proliferative LN. In particular, these were chemokines secreted in response to TNF, IL-1, and IFN-γ. The enrichment FDR (GSEA rank permutation) was <5% for all pathways except for “Natural killer activation” (16%).

FIG. 3 shows the urinary biomarkers predicting histological nephritis activity. (A) Pearson's correlations of the urinary abundance of 1000 proteins and the histological NIH Activity Index in near or same day renal biopsies. Each dot represents a protein within the array. The dashed line marks the significance threshold after correcting for multiple comparisons (FDR 10%). The area of the dot is proportional to the absolute of the correlation coefficient. Three proteins showed an FDR <10%. The FDR of IL-16 was 1.2%. Scatter plots displaying the Pearson's r and p values of correlations of the urinary abundance of IL-16, CD163, and TGF-β1 with the NIH Activity Score (B-D), NIH Chronicity Score (E-G), and proteinuria (H-J).

FIG. 4 shows that the biomarkers associated with nephritis activity decrease in responders. Urinary concentration of all biomarkers was measured at time of biopsy (WO) and after 12, 24, and 52 weeks. Thin lines depict the trajectories of each patient categorized based on the response status determined at week 52. Thick lines represent the average for each group. The urinary concentration of the 3 biomarkers significantly correlated with histological activity declined in complete and partial responders but not in non-responders (A-C). In contrast, 3 biomarkers that did not correlate with histological activity (Pearson's r range −0.0018-0.0015, p=ns) did not show a decline over time.

FIG. 5 shows the high expression of IL-16 in lupus nephritis kidney. (A) UMAP plot of scRNA-seq of renal biopsies (3131 cell) by lineage. (B) Feature plot displaying IL-16 expression at single cell level. Violin (C) and bar (D) plots summarizing the expression of the genes coding for the urinary proteins associated with nephritis activity. IL-16 was abundantly expressed by most kidney infiltrating immune cells, CD163 mostly by macrophages, and TGFB1 by NK cells. (E) Prevalence (%) of cytokine positive cells out of a compendium of 237 cytokines ranked decreasingly (top 20 are shown). IL-16 (in red) was the second most expressed cytokine in LN kidneys.

FIG. 6 shows that IL-16 positive cells are abundant in proliferative LN and qualitatively correlate with urinary IL-16 and LN activity. Immunohistochemical (IHC) staining of human IL-16 were performed in 7 LN kidney biopsies with matching urine IL-16 collected at or near the time of biopsy. The corresponding urinary abundance of IL-16 (A) and NIH Activity Index (B) of the patients whose biopsy depicted in C are plotted according to ISN class. Lower case letters (“a” to “g”) in A, B, and C identify information from the same patients. (C) IHC of IL-16 in 4 proliferative LN biopsy sections (a-d) and 3 pure membranous LN (e-g). Abundance of IL-16+ cells was noted in proliferative LN (C, a-d) with qualitatively more prominent intraglomerular IL-16 positivity in patients with higher urinary IL-16 and NIH Activity Index. Images are magnified 33.6×. Lower magnification images with larger representation of the interstitium are displayed in FIG. 11.

FIG. 7 shows the pathways enriched in the urine proteomic profiles of lupus nephritis. Pathway enrichment analysis (GO Biological Process) of the 273 urinary proteins differentially more abundant in LN revealed 12 nonoverlapping enriched pathways. Enrichment was calculated using Fischer exact test. Nonoverlapping pathway were defined using a Szymkiewicz-Simpson overlap coefficient <0.5.

FIG. 8 shows the different sources of variability in all LN patients vs healthy controls as compared to proliferative vs membranous LN. Z scores of the differential abundance of urinary proteins (defined as fold change) in all LN patients vs healthy controls (y axis) and proliferative vs membranous LN (x axis). Values are shown before (A) and after (B) centering z scores on 0.

FIG. 9 shows the pathways associated with histological NIH Activity Index. Pathway enrichment analysis (GSEA—GO Biological Process) performed on 1000 urinary proteins ranked by their correlation with histological lupus nephritis activity. All terms with FDR <10% are shown. Most pathways are involved in immune activation or chemotaxis.

FIG. 10 shows the performance IL-16, CD163, and TGF-β1 to diagnose proliferative lupus nephritis. Receiver operating characteristic curves show that urinary IL-16 has the best performance to predict proliferative lupus nephritis. The area under the curve (AUC) and relative p value were calculated using a logistic regression model and the pROC R package.

FIG. 11 shows that IL-16 is expressed by nonimmune cells in the healthy kidney. (A) Relative expression of IL-16 based on scRNA-seq (4,487 cells) of renal tissue from renal allograft rejection biopsies² revealed that infiltrating immune cells are the major source of IL-16. In the healthy kidney, snRNA-seq (4,524 nuclei)³ (B) and ATAC-seq (27,034 cells)⁴ (C), IL-16 was mostly expressed by proximal tubular epithelial and endothelial cells but also by podocytes, fibroblast and mesangial cells. Images were generated on http://humphreyslab.com/SingleCell/ and reported with Dr Benjamin Humphreys explicit permission. Cell types included proximal tubule (PT), parietal epithelial cells (PEC), loop of Henle (TAL), distal tubule (DCT1, DCT2), connecting tubule (CNT), collecting duct (PC, ICA, ICB), endothelial cells (ENDO), glomerular cell types (MES, PODO), fibroblasts (FIB), and a small population of leukocytes (LEUK).

FIG. 12 shows the tissue distribution of IL-16+ cells in LN kidney biopsies. Immunohistochemical (IHC) stainings of human IL-16 were performed in 7 kidney biopsies with matching urine IL-16 collected at or near the time of biopsy (A-G) and one class I LN biopsy used as negative control (H). In patients with more active proliferative LN there was ample interstitial (especially periglomerular) and intraglomerular IL-16 positivity. Examples of intraglomerular (green arrowheads), periglomerular (red arrows), and tubulointerstitial IL-16+ cells are portrayed in panel A. Images are magnified 10×. The corresponding higher magnification images centered on a representative glomerulus are displayed in FIG. 6 and can be identified by matching lower case letters (“a” to “g”).

FIG. 13 shows the correlation between urinary IL-16 and the NIH Activity index in an independent validation cohort of 101 patients. Scatter plot displaying the Pearson's r and p values of correlations of the urinary abundance of IL-16 (pg of IL16/mg urinary creatinine, log-transformed) with the NIH Activity Score (n=101). This independent validation cohort included 39 proliferative, 32 mixed, and 30 pure membranous LN.

FIG. 14 shows the validation of urinary IL-16 quantification by ELISA. IL-16 was quantified in 17 urinary samples using either the Kiloplex Quantibody assay or a PCR-based immunoquantitative ELISA (IQH). FIG. 14A displays the positive correlation between the 2 modalities (Pearson r 0.66, p=0.0037). FIG. 14B displays the Bland-Altman plot. The black line represents the global mean difference 0.54. Red dashed lines are positioned at 1.96 standard deviations from the mean difference.”

FIG. 15 shows the urinary biomarkers associated with the LN NIH Activity Index as described in Example 2. Volcano plots displaying Spearman correlation coefficient for 1000 (left) and 1200 (right) urinary biomarkers with LN histological activity. FDR=false discovery rate.

FIG. 16 shows that urinary IL-16 declined early in treatment responders. Longitudinal trajectories of urinary IL-16 in patients with class III, IV, or V LN (n=351) according to their response status. Thick lines connect the medians at each time point. Response was defined at 52 weeks from renal biopsy (Complete, urine pr/cr (UPCR)<0.5, serum creatinine <125% of baseline, prednisone <10 mg/day; Partial, UPCR <50% from baseline but >0.5, same creatinine and prednisone requirements; Non responder, not meeting previous definitions).

FIG. 17 shows the intrarenal expression of IL16 based on single cell RNA sequencing of lupus nephritis kidney biopsies. (A) UMAP plot showing IL16 expression by all kidney infiltrating immune cells. (B) Percentage of cytokine positive cells across all kidney infiltrating immune cells. The bar plot shows the top 25 cytokines out of a comprehensive list of 236 obtained from the “Cytokine Registry” (Immport) and the Gene Ontology database.

FIG. 18 shows the urine proteomic profile of proliferative LN as described in Example 3. (A) Volcano plot displaying the log fold change (FC) and adjusted p values of the differential abundance of 1200 urinary proteins. (B) Pathway enrichment analysis (Gene Ontology and Reactome) of the proteins enriched (FDR <1%) in proliferative LN. Odds ratios based on the hypergeometric test are displayed.

FIG. 19 shows that higher urinary neutrophil signature is associated with higher lupus nephritis activity. Heatmap of the urinary protein differentially abundant in proliferative LN. Hierarchical clustering based on protein abundance identified 3 groups. Proteinuria in mg_{protein/mg creatinine}.

FIG. 20 shows the neutrophil infiltrate in proliferative lupus nephritis. Immunofluorescence imaging of glomerular (A) and tubulointerstitial (B) neutrophil infiltration in a patient with class IV lupus nephritis. Myeloperoxidase (MPO) in green and DAPI in blue.

FIG. 21 shows the experimental pipeline as described in Example 4.

FIG. 22 shows the proteomic signatures of LN histological classes. Volcano plots of the changes of the urinary proteomic profiles of treatment responders at 3 (A), 6 (B), and 12 months (C) after kidney biopsy/treatment compared to baseline at time of biopsy. Volcano plots of the differential urinary protein abundances in pure proliferative (A), mixed (B), and membranous (C) LN compared to healthy controls (HD). Pathway enrichment analysis of the proteins enriched in pure proliferative (D), mixed (E), and membranous (F) (FDR <5%); pathways in gray had a q value >0.05. (G) Venn diagram summarizing the shared significantly changed proteins at enriched in the 3 classes displayed in A-C. (H) Heatmap summarizing the pathways enriched (FDR <25%) in the 3 classes. (I) Volcano plot displaying the differential urine protein abundances in any proliferative (pure or mixed) and pure membranous with relative pathway enrichment analysis (J). (K) Heatmap displaying the unsupervised clustering based the urine abundances of the proteins differentially abundant in any proliferative vs pure membranous (panel I); clinical features are displayed.
FDR=false discovery rate; q=adjusted p value (Benjamini-Hochberg).

FIG. 23 shows the proteomic signatures of histological activity and chronicity. Volcano plots displaying Pearson's correlation of the proteins urinary abundances and the NIH Activity (A) and Chronicity (C) indices. The correlation with the urine protein-to-creatinine ratio (UPCR) is indicated for reference. Pathway enrichment analysis (by GSEA) of the associations of the urinary proteins with the NIH Activity (B) and Chronicity (D) indices. FDR=false discovery rate; q=Benjamini-Hochberg adjusted p value.

FIG. 24 shows the proteomic changes of treatment response. Volcano plots of the changes of the urinary proteomic profiles of treatment responders at 3 months after kidney biopsy/treatment compared to baseline at time of biopsy in proliferative and membranous combined (A) or proliferative only (B). (C and D) Pathway enrichment analysis of the urinary protein declined in A and B, respectively. (E) Venn diagram summarizing the shared significantly changed proteins at the 3, 6, and 12 months after the kidney biopsy. (F) Heatmap displaying the urinary abundances of the proteins significantly decreased at 3 months in responders (panel A) at the 4 time points according to response status. (G) Discriminatory power of the change of each urinary protein at 3 months compared to baseline to predict treatment response at month 12 (displayed as area under the curve, AUC). The change in urine protein-to-creatinine ratio (UPCR) is displayed for refence as the traditionally used biomarker. (H) Receiver operating characteristic curves of the decline at 3 months of the UPCR (traditional biomarker) and urinary CD163. I and J replicate G and H, but limited to patients with proliferative LN. (K) Trajectory of the urinary abundance of CD163 (K) and CD206 (L) according to response status in all patients and stratified by ISN class. Thin lines indicate individual trajectories; thick lines indicate the group medians.
q values=adjusted p values (Benjamini-Hochberg). OR=odds ratio. FDR=false discovery rate.

FIG. 25 shows GFR trajectories of LN patients as described in Example 5. Individual GFR starting at time of diagnostic kidney biopsy trajectories are displayed. Treatment was at the discretion of the treating physician. GFR expressed as change from the baseline value. “Loss” was defined if >15 ml/min of GFR was lost by year 3.

FIG. 26 shows that persistent elevation of biomarkers of LN activity predicts at 1 year predict GFR loss at 3 years. Urinary abundances at 1 year from the diagnostic kidney biopsy of pre-specified biomarkers are displayed. Proteinuria (UPCR) is also displayed for comparison as the current clinical standard.

### DETAILED DESCRIPTION

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Analyte” as used herein refers to any component of a biological sample that is desired to be detected (such as, for example, IL-16, CD163, Galectin-1, proteinase 3, or any combination thereof). The term can be used to refer to a single component or a sample or a plurality of components in a sample.

“Antibody” and “antibodies” as used herein refers to monoclonal antibodies, monospecific antibodies (e.g., which can either be monoclonal, or may also be produced by other means than producing them from a common germ cell), bi-specific or multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” or “antigen-binding fragment” as used interchangeably herein, refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers.

Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

A “biochip” as used herein refers to a solid substrate having a generally planar surface to which an adsorbent is attached. Frequently, the surface of a biochip comprises a plurality of addressable locations, each of which location has the adsorbent bound there. Biochips can be adapted to engage a probe interface, and therefore, function as probes.

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, a monoclonal antibody, a chimeric antibody, a CDR-grafted antibody, a humanized antibody, an affinity matured antibody, and fragments of any such antibodies that retain the ability to bind to an antigen. In other aspects, a binding protein can be an aptamer, such as a nucleic acid, that can selectively bind to a specific target.

As used herein, “CD163” refers to the hemoglobin (Hb) scavenger receptor, a macrophage-specific protein. The upregulated expression of CD163 is one of the major changes in the macrophage switch to alternative activated phenotypes in inflammation. CD163 is a 130-kDa membrane protein with a short cytoplasmic tail, a single transmembrane segment, and a large ectodomain consisting of nine scavenger receptor cysteine-rich (SRCR) scavenger receptor class B domains (62). Different isoforms of human CD163 have been described, including three variants with different length of the cytoplasmic tail (62), with the short tail form (42 amino acids) being the most abundant. All variants contain common internalization motifs and exhibit endocytic activity (62).

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like). As used herein, a “control subject” relates to a subject or subjects that has does not have lupus nephritis or that does not have proliferative lupus nephritis.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Detecting the presence of” as used herein refers to the qualitative measurement of one or more compounds or biomarkers (e.g., IL-16, Galectin-1, CD163, CD206, FOLR2, proteinase 3 or any combination thereof) in a biological sample obtained from a subject.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

As used herein, the term “Galectin-1” or “GAL1” refers to the first identified member of the galectin family. Galectins are a phylogenetically conserved family of lectins and share consensus of amino-acid-sequences of about 130 amino acids and a carbohydrate recognition domain (CRD) responsible for β-galactoside binding. Galectins have been found to be abundantly expressed by many cell types, such as skeletal, smooth and cardiac muscle and from other cells of mesenchymal origin. The human amino acid and nucleic acid sequence for Galectin-1 can be found in GenBank Accession No. P09382.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

As used herein, “IL-16” refers to a pro-inflammatory cytokine that is chemotactic for CD4+T lymphocytes, monocytes, and eosinophils. In addition to inducing chemotaxis, IL-16 can upregulate IL-2 receptor and HLA-DR4 expression, inhibit T cell receptor (TcR)/CD3-dependent activation, and promote repression of HIV-1 transcription. IL-16 is a unique cytokine with no significant sequence homology to other well-characterized cytokines or chemokines. The human amino acid and nucleic acid sequence for IL-16 can be found in GenBank Accession No. Q14005.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence. As used herein, “isolated polypeptide” refers to a polypeptide (e.g., of recombinant, synthetic or chemical original or a combination thereof), that, by virtue of its origin, the isolated polypeptide is not associated with all or a portion of a polypeptide and/or other protein(s) with which the “isolated polypeptide” is found in nature; is operably linked to a polypeptide and/or protein that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 1251, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon.

As used herein, “marker” refers to a polypeptide (of a particular apparent molecular weight), which is differentially present in a sample taken from patients having lupus nephritis as compared to a comparable sample taken from control subjects. The term “biomarker” is used interchangeably with the term “marker.”

As used herein, “proteinase 3” or “PRTN3” refers to a 29,000 Da neutral serine proteinase stored in the azurophil granules of polymorphonuclear leukocytes. PRTN3 has broad proteolytic activity and degrades a variety of extracellular matrix proteins, including fibronectin, type IV collagen and laminin. The human amino acid and nucleic acid sequence for PRTN3 can be found in GenBank Accession No. P24158.

“Reference level” as used herein refers to an assay cutoff value (or level) that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). As used herein, the term “cutoff” refers to a limit (e.g., such as a number) above which there is a certain or specific clinical outcome and below which there is a different certain or specific clinical outcome.

“Sample,” “test sample,” “specimen,” “sample from a subject,” “biological sample,” and “patient sample” may be used interchangeably herein to refer to a sample of blood, such as whole blood (including for example, capillary blood, venous blood, dried blood spot, etc.), saliva, tissue, urine, serum, plasma, tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner, or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter plate or well, stick, bead (including a microbead), microparticle, biochip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

“Specific binding partner” or “Specific binding member,” as used interchangeable herein, is a member of a specific binding pair that exhibit specific binding. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human). In some aspects, the subject may be a human, a non-human primate or a cat. In some aspects, the subject is a human. In some aspects, the subject is suspected of having lupus nephritis. In some aspects, the subject is a human who has systemic lupus erythematosus, who may or may be suspected of having lupus nephritis or proliferative lupus nephritis. In other aspects, the subject is a human who has systemic lupus erythematosus and has a history of lupus nephritis or proliferative lupus nephritis. In other aspects, the subject is a human who has systemic lupus and does not have a history of lupus nephritis or proliferative lupus nephritis. In yet other aspects, the subject is a human who has systemic lupus erythematosus, who may or may not have lupus nephritis or proliferative lupus nephritis, and does not have proteinuria. In still other aspects, the subject is a human who has systemic lupus erythematous, who may or may not have lupus nephritis or proliferative lupus nephritis, and has proteinuria. In other aspects, the subject is a human who has systemic lupus erythematosus, a history of lupus nephritis or proliferative lupus nephritis, and has proteinuria. In other aspects, the subject is a human who has systemic lupus, does not have a history of lupus nephritis or proliferative lupus nephritis, and has proteinuria. In other aspects, the subject is a human who has systemic lupus erythematosus, a history of lupus nephritis or proliferative lupus nephritis, and does not have proteinuria. In other aspects, the subject is a human who has systemic lupus, does not have a history of lupus nephritis or proliferative lupus nephritis, and does not have proteinuria. In other aspects, the subject or patient may be undergoing treatment.

As used herein, a “system” refers to a plurality of real and/or abstract elements operating together for a common purpose. In some aspects, a “system” is an integrated assemblage of hardware and/or software elements. In some aspects, each component of the system interacts with one or more other elements and/or is related to one or more other elements. In some aspects, a system refers to a combination of components and software for controlling and directing methods.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” is used herein to describe a peptide or polypeptide that differs from a reference peptide or polypeptide in amino acid sequence by the insertion, deletion, or conservative substitution of amino acids, but retains at least one biological activity. Representative examples of “biological activity” include the ability to be bound by a specific antigen or antibody, or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically-reactive fragment of an anti-analyte antibody that differs from the corresponding fragment of anti-analyte antibody in amino acid sequence but is still antigenically reactive and can compete with the corresponding fragment of anti-analyte antibody for binding with the analyte. “Variant” also can be used to describe a polypeptide or a fragment thereof that has been differentially processed, such as by proteolysis, phosphorylation, or other post-translational modification, yet retains its antigen reactivity.

### 2. Methods of Diagnosis Lupus Nephritis, Predicting the Risk of Developing Lupus Nephritis and Response to Treatment for Lupus Nephritis

In one embodiment, the present disclosure relates to methods of diagnosing lupus nephritis, such as, proliferative lupus nephritis, in a subject. The method involves obtaining at least one biological sample from a subject. The time period in which the sample is obtained from the subject is not critical. Once at least one sample is obtained from the subject, the presence of at least one of IL-16, Galectin-1, CD163, CD206, FOLR2, proteinase 3, or a combination thereof is determined or detected in the sample using routine techniques known in the art. In some aspects, at least two of IL-16, Galectin-1, CD163, CD206, FOLR2, and/or proteinase 3 are determined in the sample. In yet other aspects, at least three of IL-16, Galectin-1, CD163, CD206, FOLR2, and/or proteinase 3 are determined in the sample. In yet another aspect, each of IL-16, Galectin-1, CD163, CD206, FOLR2, and proteinase 3 is determined in the sample. Optionally, the above method can further involve determining the presence of at least one additional biomarker from Table 1 from a subject.

TABLE 1

| | Activity | | | | |
|---|---|---|---|---|---|
| | Index | False | | | |
| | Correlation | discovery | | | |
| Target | P value | rate | UniProt | Gene Name | Gene ID |
| IL-16 | 2.80E−10 | 3.40E−07 | Q14005 | IL16 | 3603 |
| CD163 | 2.90E−09 | 1.70E−06 | Q86VB7 | CD163 M130 | 9332 |
| PRTN3 | 2.20E−08 | 8.80E−06 | P24158 | PRTN3 MBN | 5657 |
| Cyclophilin | 4.70E−08 | 1.40E−05 | P62937 | PPIA CYPA | 5478 |
| A | | | | | |
| S100A13 | 8.80E−08 | 2.10E−05 | Q99584 | S100A13 | 6284 |
| Galectin-1 | 1.80E−07 | 3.40E−05 | P09382 | LGALS1 | 3956 |
| FKBP51 | 2.00E−07 | 3.40E−05 | Q13451 | FKBP5 AIG6 FKBP51 | 2289 |
| Catalase | 3.80E−07 | 5.70E−05 | P04040 | CAT | 847 |
| MMR | 4.40E−07 | 5.80E−05 | P22897 | MRC1 CLEC13D | 4360 |
| | | | | CLEC13DL MRC1L1 | |
| CES1 | 1.00E−06 | 0.00011 | P23141 | CES1 CES2 SES1 | 1066 |
| MMP-8 | 1.10E−06 | 0.00011 | P22894 | MMP8 CLG1 | 4317 |
| S100A8 | 1.10E−06 | 0.00011 | P05109 | S100A8 CAGA CFAG | 6279 |
| | | | | MRP8 | |
| Caspase-3 | 1.60E−06 | 0.00015 | P42574 | CASP3 CPP32 | 836 |
| Attractin | 1.80E−06 | 0.00015 | O75882 | ATRN KIAA0548 MGCA | 8455 |
| TSP-1 | 2.50E−06 | 2.00E−04 | P07996 | THBS1 TSP TSP1 | 7057 |
| Glypican 1 | 3.10E−06 | 0.00023 | P35052 | GPC1 | 2817 |
| LEDGF | 3.30E−06 | 0.00023 | O75475 | PSIP1 DFS70 LEDGF | 11168 |
| | | | | PSIP2 | |
| Cathepsin S | 3.70E−06 | 0.00025 | P25774 | CTSS | 1520 |
| Nidogen-1 | 4.90E−06 | 0.00031 | P14543 | NID1 NID | 4811 |
| NCAM-1 | 6.60E−06 | 0.00039 | P13591 | NCAM1 NCAM | 4684 |
| FAP | 7.20E−06 | 0.00041 | Q12884 | FAP | 2191 |
| Annexin V | 1.70E−05 | 0.00093 | P08758 | ANXA5 ANX5 ENX2 PP4 | 308 |
| MCP-1 | 2.70E−05 | 0.0014 | P13500 | CCL2 MCP1 SCYA2 | 6347 |
| MIP-1b | 3.60E−05 | 0.0018 | P13236 | CCL4 LAG1 MIP1B | 388372; 6351 |
| | | | | SCYA4 | |
| TIM-1 | 4.00E−05 | 0.0019 | Q96D42 | HAVCR1 KIM1 TIM1 | 26762 |
| | | | | TIMD1 | |
| EGF R | 4.20E−05 | 0.0019 | P00533 | EGFR ERBB ERBB1 | 1956 |
| | | | | HER1 | |
| CD36 | 4.60E−05 | 0.002 | P16671 | CD36 GP3B GP4 | 948 |
| Osteoactivin | 5.70E−05 | 0.0024 | Q14956 | GPNMB HGFIN NMB | 10457 |
| | | | | UNQ1725/PRO9925 | |
| Tenascin C | 6.30E−05 | 0.0026 | P24821 | TNC HXB | 3371 |
| TIM-4 | 6.50E−05 | 0.0026 | Q96H15 | TIMD4 TIM4 | 91937 |
| Visfatin | 7.80E−05 | 0.003 | P43490 | NAMPT PBEF PBEF1 | 10135 |
| Granzyme A | 8.20E−05 | 0.0031 | P12544 | GZMA CTLA3 HFSP | 3001 |
| FCRL3 | 8.80E−05 | 0.0032 | Q96P31 | FCRL3 FCRH3 IFGP3 | 115352 |
| | | | | IRTA3 SPAP2 | |
| IL-6 | 9.00E−05 | 0.0032 | P05231 | IL6 IFNB2 | 3569 |
| Pentraxin 3 | 9.50E−05 | 0.0033 | P26022 | PTX3 TNFAIP5 TSG14 | 5806 |
| TLR2 | 0.00013 | 0.0043 | O60603 | TLR2 TIL4 | 7097 |
| IDS | 0.00013 | 0.0043 | P22304 | IDS SIDS | 3423 |
| Midkine | 0.00017 | 0.0054 | P21741 | MDK MK1 NEGF2 | 4192 |
| CPM | 0.00018 | 0.0054 | P14384 | CPM | 1368 |
| OPG | 2.00E−04 | 0.006 | O00300 | TNFRSF11B OCIF OPG | 4982 |
| SLPI | 0.00022 | 0.0063 | P03973 | SLPI WAP4 WFDC4 | 6590 |
| Desmoglein | 0.00025 | 0.0072 | Q14126 | DSG2 CDHF5 | 1829 |
| 2 | | | | | |
| bIG-H3 | 0.00026 | 0.0073 | Q15582 | TGFBI BIGH3 | 7045 |
| IGSF4B | 0.00027 | 0.0073 | Q8N126 | CADM3 IGSF4B NECL1 | 57863 |
| | | | | SYNCAM3 TSLL1 | |
| | | | | UNQ225/PRO258 | |
| PCSK9 | 0.00028 | 0.0076 | Q8NBP7 | PCSK9 NARC1 | 255738 |
| | | | | PSEC0052 | |
| MIP-1a | 0.00029 | 0.0076 | P10147 | CCL3 GOS19-1 MIP1A | 6348 |
| | | | | SCYA3 | |
| DBH | 0.00031 | 0.008 | P09172 | DBH | 1621 |
| ESAM | 0.00037 | 0.0091 | Q96AP7 | ESAM UNQ220/PRO246 | 90952 |
| Azurocidin | 0.00038 | 0.0091 | P20160 | AZU1 | 566 |
| PDGF-AA | 0.00039 | 0.0091 | P04085 | PDGFA PDGF1 | 5154 |
| LAP(TGFb1) | 0.00039 | 0.0091 | P01137.2 | TGFB1 | 7040 |
| HO-1 | 4.00E−04 | 0.0091 | P09601 | HMOX1 HO HO1 | 3162 |
| TSP-4 | 0.00043 | 0.0098 | P35443 | THBS4 TSP4 | 7060 |
| LAMP1 | 5.00E−04 | 0.011 | P11279 | LAMP1 | 3916 |
| Integrin | 5.00E−04 | 0.011 | P56199 | ITGA1 | 3672 |
| alpha 1 | | | | | |
| NQO-1 | 0.00054 | 0.011 | P15559 | NQO1 DIA4 NMOR1 | 1728 |
| | 0.00055 | 0.011 | Q9BXN2 | CLEC7A BGR CLECSF12 | 64581 |
| | | | | DECTIN1 | |
| Dectin-1 | | | | UNQ539/PRO1082 | |
| Contactin-4 | 0.00055 | 0.011 | Q8IWV2 | CNTN4 | 152330 |
| Neuropilin-1 | 0.00056 | 0.011 | O14786 | NRP1 NRP VEGF165R | 8829 |
| CrkL | 0.00057 | 0.011 | P46109 | CRKL | 1399 |
| Syndecan-4 | 6.00E−04 | 0.012 | P31431 | SDC4 | 6385 |
| CES2 | 0.00061 | 0.012 | O00748 | CES2 ICE | 8824 |
| LAMP2 | 0.00062 | 0.012 | P13473 | LAMP2 | 3920 |
| MBL | 0.00069 | 0.013 | P11226 | MBL2 COLEC1 MBL | 4153 |
| IGFBP-1 | 7.00E−04 | 0.013 | P08833 | IGFBP1 IBP1 | 3484 |
| Dkk-3 | 0.00071 | 0.013 | Q9UBP4 | DKK3 REIC | 27122 |
| | | | | UNQ258/PRO295 | |
| TFPI | 0.00076 | 0.014 | P10646 | TFPI LACI TFPI1 | 7035 |
| Angiostatin | 0.00079 | 0.014 | P00747 | PLG | 5340 |
| LIMPII | 0.00085 | 0.015 | Q14108 | SCARB2 CD36L2 LIMP2 | 950 |
| | | | | LIMPII | |
| CEACAM-1 | 0.00088 | 0.015 | P13688 | CEACAM1 BGP BGP1 | 634 |
| TAFA5 | 0.00097 | 0.016 | Q7Z5A7 | FAM19A5 TAFA5 | 25817 |
| | | | | UNQ5208/PRO34524 | |
| RANTES | 0.001 | 0.017 | P13501 | CCL5 D17S136E SCYA5 | 6352 |
| MCSF R | 0.0013 | 0.02 | P07333 | CSFIR FMS | 1436 |
| AFP | 0.0013 | 0.02 | P02771 | AFP HPAFP | 174 |
| MMP-9 | 0.0013 | 0.02 | P14780 | MMP9 CLG4B | 4318 |
| AMICA | 0.0013 | 0.02 | Q86YT9 | JAML AMICA1 | 120425 |
| | | | | UNQ722/PRO1387 | |
| GP73 | 0.0013 | 0.02 | Q8NBJ4 | GOLM1 C9orf155 | 51280 |
| | | | | GOLPH2 PSEC0242 | |
| | | | | UNQ686/PRO1326 | |
| Serpin B6 | 0.0013 | 0.02 | P35237 | SERPINB6 PI6 PTI | 5269 |
| Kynureninase | 0.0013 | 0.02 | Q16719 | KYNU | 8942 |
| C1qTNF1 | 0.0013 | 0.02 | Q9BXJ1 | C1QTNF1 CTRP1 | 114897 |
| | | | | UNQ310/PRO353 | |
| IL-6R | 0.0014 | 0.02 | P08887 | IL6R | 3570 |
| GROa | 0.0014 | 0.021 | P09341 | CXCL1 GRO GRO1 | 2919 |
| | | | | GROA MGSA SCYB1 | |
| SHP-1 | 0.0014 | 0.02 | P29350 | PTPN6 HCP PTP1C | 5777 |
| VSIG4 | 0.0015 | 0.022 | Q9Y279 | VSIG4 CRIg Z39IG | 11326 |
| | | | | UNQ317/PRO362 | |
| GLA | 0.0017 | 0.024 | P06280 | NA | 2717 |
| CD97 | 0.0018 | 0.025 | P48960 | CD97 | 976 |
| CD34 | 0.0018 | 0.025 | P28906 | CD34 | 947 |
| htPAPP-A | 0.0021 | 0.029 | Q13219/P13727 | PAPPA | 5069; 5553 |
| CPA1 | 0.0022 | 0.029 | P15085 | CPA1 CPA | 1357 |
| aFGF | 0.0024 | 0.032 | P05230 | FGF1 FGFA | 2246 |
| Serpin B8 | 0.0024 | 0.032 | P50452 | SERPINB8 PI8 | 5271 |
| TSH | 0.0025 | 0.033 | P01222/P01215 | TSHB/CGA | 7252; 1081 |
| MOG | 0.0025 | 0.033 | Q16653 | MOG | 4340 |
| GHR | 0.0027 | 0.034 | P10912 | GHR | 2690 |
| LRPAP | 0.0027 | 0.034 | P30533 | A2MRAP LRPAP1 | 4043 |
| Park7 | 0.0027 | 0.034 | Q99497 | PARK7 | 11315 |
| DLL1 | 0.0028 | 0.034 | O00548 | DLL1 UNQ146/PRO172 | 28514 |
| ROBO2 | 0.0028 | 0.034 | Q9HCK4 | ROBO2 KIAA1568 | 6092 |
| CXCL14 | 0.0029 | 0.035 | 095715 | CXCL14 MIP2G NJAC | 9547 |
| | | | | SCYB14 PSEC0212 | |
| | | | | UNQ240/PRO273 | |
| LAMA4 | 0.0029 | 0.035 | Q16363 | LAMA4 | 3910 |
| PRDX4 | 0.003 | 0.036 | Q13162 | PRDX4 | 10549 |
| SULT2B1 | 0.0031 | 0.036 | O00204 | SULT2B1 HSST2 | 6820 |
| Cadherin-11 | 0.0032 | 0.037 | P55287 | CDH11 | 1009 |
| Activin RIB | 0.0034 | 0.039 | P36896 | ACVR1B ACVRLK4 | 91 |
| | | | | ALK4 | |
| PILR-alpha | 0.0034 | 0.039 | Q9UKJ1 | PILRA | 29992 |
| CANT1 | 0.0035 | 0.039 | Q8WVQ1 | CANT1 SHAPY | 124583 |
| Hepassocin | 0.0035 | 0.039 | Q08830 | FGL1 HFREP1 | 2267 |
| Hepsin | 0.0036 | 0.04 | P05981 | HPN TMPRSS1 | 3249 |
| Trypsin Pan | 0.0036 | 0.04 | P07478 | PRSS2 TRY2 TRYP2 | 5645 |
| Cripto-1 | 0.0037 | 0.04 | P13385 | TDGF1 CRIPTO | 6997 |
| Decorin | 0.0037 | 0.04 | P07585 | DCN SLRR1B | 1634 |
| C1q R1 | 0.0042 | 0.045 | Q9NPY3 | CD93 C1QR1 MXRA4 | 22918 |
| Endoglin | 0.0044 | 0.047 | P17813 | ENG END | 2022 |
| BLMH | 0.0044 | 0.047 | Q13867 | BLMH | 642 |
| LRIG3 | 0.0045 | 0.047 | Q6UXM1 | LRIG3 LIG3 | 121227 |
| | | | | UNQ287/PRO326/PRO335 | |
| Pepsinogen II | 0.0046 | 0.047 | P20142 | PGC | 5225 |
| CXCL16 | 0.0047 | 0.047 | Q9H2A7 | CXCL16 SCYB16 | 58191 |
| | | | | SRPSOX | |
| | | | | UNQ2759/PRO6714 | |
| METAP2 | 0.0047 | 0.047 | P50579 | METAP2 MNPEP | 10988 |
| | | | | P67EIF2 | |
| GITR L | 0.0047 | 0.047 | Q9UNG2 | TNFSF18 AITRL GITRL | 8995 |
| | | | | TL6 UNQ149/PRO175 | |
| IL-1 R3 | 0.0047 | 0.047 | Q9NPH3 | IL1RAP C3orf13 IL1R3 | 3556 |
| Nestin | 0.005 | 0.049 | P48681 | NES Nbla00170 | 10763 |
| p27 | 0.0051 | 0.05 | P46527 | CDKN1B KIP1 | 1027 |
| NRG1-b1 | 0.0057 | 0.055 | AAA58639 | NRG1 | 3084 |
| Cathepsin C | 0.0057 | 0.055 | P53634 | CPPI CTSC | 1075 |
| Fcg RIIBC | 0.0061 | 0.058 | P31994/P31995 | FCGR2B | 2213; 9103 |
| PCSK2 | 0.0061 | 0.058 | P16519 | PCSK2 NEC2 | 5126 |
| Syntaxin 4 | 0.0064 | 0.06 | Q12846 | STX4 STX4A | 6810 |
| MAGP-2 | 0.0064 | 0.06 | Q13361 | MFAP5 MAGP2 | 8076 |
| C5a | 0.0069 | 0.064 | P01031 | C5 CPAMD4 | 727 |
| CA125 | 0.0071 | 0.065 | Q8WXI7 | MUC16 CA125 | 94025 |
| Contactin-1 | 0.0072 | 0.065 | Q12860 | CNTN1 | 1272 |
| Arginase 1 | 0.0072 | 0.065 | P05089 | ARG1 | 383 |
| ASAHL | 0.0073 | 0.065 | Q02083 | NAAA ASAHL PLT | 27163 |
| IGF-1R | 0.0074 | 0.066 | P08069 | IGF1R | 3480 |
| Spinesin | 0.0075 | 0.067 | Q9H3S3 | TMPRSS5 | 80975 |
| LEKTI | 0.0075 | 0.067 | Q9NQ38 | SPINK5 | 11005 |
| Latexin | 0.0076 | 0.067 | Q9BS40 | NA | 56925 |
| TIMP-1 | 0.0081 | 0.07 | P01033 | TIMP1 CLGI TIMP | 7076 |
| ALK-1 | 0.0085 | 0.073 | P37023 | ACVRL1 ACVRLK1 | 94 |
| | | | | ALK1 | |
| PAM | 0.0085 | 0.073 | P19021 | PAM | 5066 |
| Cystatin E M | 0.009 | 0.076 | Q15828 | CST6 | 1474 |
| CEA | 0.0093 | 0.078 | P06731 | CEACAM5 CEA | 1048 |
| C2 | 0.0094 | 0.079 | P06681 | C2 | 717 |
| FGF-21 | 0.0095 | 0.079 | Q9NSA1 | FGF21 | 26291 |
| | | | | UNQ3115/PRO10196 | |
| TIMP-2 | 0.0096 | 0.08 | P16035 | TIMP2 | 7077 |
| ErbB3 | 0.0097 | 0.08 | P21860 | ERBB3 HER3 | 2065 |
| LRRC4 | 0.0098 | 0.08 | Q9HBW1 | LRRC4 BAG NAG14 | 64101 |
| | | | | UNQ554/PRO1111 | |
| VEGF R1 | 0.01 | 0.084 | P17948 | FLT1 FLT FRT VEGFR1 | 2321 |
| HS3ST4 | 0.01 | 0.081 | Q9Y661 | HS3ST4 3OST4 | 9951 |
| Dystroglycan | 0.01 | 0.084 | Q14118 | DAG1 | 1605 |
| TACE | 0.011 | 0.084 | P78536 | ADAM17 CSVP TACE | 6868 |
| CLEC10A | 0.011 | 0.087 | Q8IUN9 | CLEC10A CLECSF13 | 10462 |
| | | | | CLECSF14 HML | |
| CD73 | 0.011 | 0.087 | P21589 | NT5E NT5 NTE | 4907 |
| PDCD6 | 0.011 | 0.087 | 075340 | ALG2 PDCD6 | 10016 |
| Persephin | 0.011 | 0.087 | O60542 | PSPN | 5623 |
| TNF RI | 0.012 | 0.09 | P19438 | TNFRSF1A TNFAR | 7132 |
| | | | | TNFR1 | |
| DR6 | 0.012 | 0.092 | 075509 | TNFRSF21 DR6 | 27242 |
| | | | | UNQ437/PRO868 | |
| HAI-2 | 0.012 | 0.09 | 043291 | SPINT2 HAI2 KOP | 10653 |
| Cystatin S | 0.012 | 0.092 | P01036 | CST4 | 1472 |
| PLUNC | 0.012 | 0.092 | Q9NP55 | BPIFA1 LUNX NASG | 51297 |
| | | | | PLUNC SPLUNC1 | |
| | | | | SPURT | |
| | | | | UNQ787/PRO1606 | |
| EG-VEGF | 0.013 | 0.093 | P58294 | PROK1 | 84432 |
| | | | | UNQ600/PRO1186 | |
| IGFBP-6 | 0.013 | 0.093 | P24592 | IGFBP6 IBP6 | 3489 |
| BAFF | 0.013 | 0.095 | Q9Y275 | TNFSF13B BAFF BLYS | 10673 |
| | | | | TALL1 TNFSF20 ZTNF4 | |
| | | | | UNQ401/PRO738 | |
| CHI3L1 | 0.013 | 0.093 | P36222 | CHI3L1 | 1116 |
| Angiogenin | 0.014 | 0.099 | P03950 | ANG RNASE5 | 283 |
| VE−Cadherin | 0.014 | 0.099 | P33151 | CDH5 | 1003 |

In some aspects, the subject, is diagnosed as having lupus nephritis, such as, proliferative lupus nephritis, if the presence of at least one of IL-16, Galectin-1, CD163, CD206, FOLR2, and/or proteinase 3 and optionally, at least one biomarker from Table 1, is detected in the sample. In other aspects, the subject is diagnosed as having lupus nephritis, such as, proliferative lupus nephritis, if the presence of at least two of IL-16, Galectin-1, CD163, CD206, FOLR2, and/or proteinase 3 and optionally, at least one biomarker from Table 1, are detected in the sample. In still yet other aspects, the subject is diagnosed as having lupus nephritis, such as, proliferative lupus nephritis, if the presence of at least three of IL-16, Galectin-1, CD163, CD206, FOLR2, and/or proteinase 3 and optionally, at least one biomarker from Table 1, are detected in the sample. In still yet further aspects, the subject is diagnosed as having lupus nephritis, such as, proliferative lupus nephritis, if the presence of each of IL-16, Galectin-1, CD163, CD206, FOLR2, and proteinase 3 and optionally, at least one biomarker from Table 1, is detected in the sample.

Once a subject is diagnosed with lupus nephritis, such as, proliferative lupus nephritis, the subject can be further treated according to routine techniques known in the art. For example, the subject can be treated with at least with at least one immunosuppressant (such as cyclosporine, tacrolimus, a calcineurin-inhibitor, cyclophosphamide, hydroxychloroquine, azathioprine, mycophenolate, or any combination thereof), at least one corticosteroid (such as prednisone, prednisolone, methylprednisolone, or any combination thereof), a B-lymphocyte stimulator specific inhibitor (such as belimumab), biologics, such as, rituximab, or any combination thereof.

In the above method, the subject can be a human. In some aspects, the subject is suspected of having lupus nephritis. In some aspects, the subject is a human who has systemic lupus erythematosus, who may or may not be suspected of having lupus nephritis, such as, proliferative lupus nephritis. In yet other aspects, the subject is a human who has systemic lupus erythematosus, who may or may not have lupus nephritis, such as, proliferative lupus nephritis, and does not have proteinuria. In still other aspects, the subject is a human who has systemic lupus erythematous, who may or may not have lupus nephritis, such as, proliferative lupus nephritis, and does have proteinuria. In yet another aspect, the sample obtained from the subject is a whole blood sample, a plasma sample, a serum sample or a urine sample. In some aspects, the sample is a whole blood sample. In other aspects, the sample is a serum sample. In yet other aspects, the biological sample is a urine sample.

In another embodiment, the present disclosure relates to methods of predicting a subject's risk of developing lupus nephritis, such as, proliferative lupus nephritis. In this aspect, the level of at least one of IL-16, Galectin-1, CD163, CD206, FOLR2, PRTN3 and optionally, at least one biomarker in Table 1, or any combination thereof is determined using any of the methods described previously herein. In some aspects of this method, the level of at least two biomarkers of IL-16, Galectin-1, CD163, CD206, FOLR2, and/or proteinase 3 and optionally, at least one biomarker from Table 1 is determined in the sample. In yet other aspects, the level of at least three biomarkers of IL-16, Galectin-1, CD163, CD206, FOLR2, and/or proteinase 3 and optionally, at least one biomarker from Table 1, is determined in the sample. In yet another aspect, the level of each of the biomarkers of IL-16, Galectin-1, CD163, CD206, FOLR2, proteinase 3 and optionally, at least one biomarker from Table 1, is determined in the sample.

Once the level of one of more of IL-16, Galectin-1, CD163, CD206, FOLR2, and/or PRTN3 and optionally, at least one biomarker from Table 1 is determined in the sample, the level is compared to a control level for the same biomarker. For example, if the level of IL-16 in a sample is determined according to the methods described herein, it is compared to a control level for IL-16. By way of another example, if the levels of each of IL-16 and Galectin-1 in a sample are determined according to the methods described herein, the level of IL-16 is compared to a control level of IL-16 and the level of Galectin-1 is compared to a control level of Galectin-1.

In some aspects, the control level(s) is the level of IL-16, Galectin-1, CD163, CD206, FOLR2, and/or PRTN3 and optionally, at least one biomarker from Table 1, in subject that not have lupus nephritis, such as, proliferative lupus nephritis. Based on the comparison, the level of the at least one IL-16, Galectin-1, CD163, CD206, FOLR2, and/or PRTN3 and optionally, at least one biomarker from Table 1, will be determined to be higher or lower than its corresponding control level. If the level of the at least one of IL-16, Galectin-1, CD163, CD206, FOLR2, and/or PRTN3 and optionally, at least one biomarker from Table 1, is higher than its corresponding control level, then the subject is determined to be at risk of developing lupus nephritis, such as, proliferative lupus nephritis. If the level of the at least one IL-16, Galectin-1, CD163, CD206, FOLR2, and/or PRTN3 and optionally, at least one biomarker from Table 1, is lower than its corresponding control level, then the subject is determined not to be at risk of developing lupus nephritis, such as, proliferative lupus nephritis.

The method described herein can be repeated as needed to continually monitor and/or assess a patient's risk of developing lupus nephritis, such as, proliferative lupus nephritis. In other words, there is no limit on the number of times the method can be performed. In particular, the method described herein can be used to monitor the activity of a lupus nephritis, such as, proliferative lupus nephritis in a subject, such as a subject suffering from systemic lupus erythematosus, who may or may not have proteinuria.

Additionally, if, as a result of the above method, it is determined that the subject is at risk of developing lupus nephritis, such as, proliferative lupus nephritis, the method can further comprise treating the patient with at least one of the treatments described previously herein to try and prevent the onset of lupus nephritis, such as, proliferative lupus nephritis. In the above method, the subject can be a human. In some aspects, the subject is suspected of having lupus nephritis, such as, proliferative lupus nephritis. In some aspects, the subject is a human who has systemic lupus erythematosus, who may or may be suspected of having lupus nephritis, such as, proliferative lupus nephritis. In yet other aspects, the subject is a human who has systemic lupus erythematosus, who may or may not have lupus nephritis, such as, proliferative lupus nephritis, and does not have proteinuria. In still other aspects, the subject is a human who has systemic lupus erythematous, who may or may not have lupus nephritis, such as, proliferative lupus nephritis, and does have proteinuria.

In yet another aspect, the sample obtained from the subject is a whole blood sample, a plasma sample, a serum sample or a urine sample. In some aspects, the sample is a whole blood sample. In other aspects, the sample is a serum sample. In yet other aspects, the biological sample is a urine sample.

In yet another embodiment, the present disclosure relates to a method of determining whether a subject suffering from lupus nephritis, such as, proliferative lupus nephritis, is responding to treatment for said lupus nephritis. In this aspect, a biological sample is obtained from a subject being treated for lupus nephritis, such as, proliferative lupus nephritis. In some aspects, the subject may be being treated with one of treatments described previously herein. In other aspects, the subject may be being treated with other treatments other than those described herein. Additionally, in some aspects, the subject has systemic lupus erythematous with proteinuria. In other aspects, the subject has systemic lupus erythematous without proteinuria.

In some aspects, the sample in the above method is obtained from the subject suffering from lupus nephritis, such as, proliferative lupus nephritis at about 3 months after kidney treatment. In some aspects, the sample in the above method is obtained from the subject at about 4 months after kidney treatment. In some aspects, the sample in the above method is obtained from the subject at about 5 months after kidney treatment. In some aspects, the sample in the above method is obtained from the subject at about 6 months after kidney treatment. In some aspects, the sample in the above method is obtained from the subject at about 7 months after kidney treatment. In some aspects, the sample in the above method is obtained from the subject at about 8 months after kidney treatment. In some aspects, the sample in the above method is obtained from the subject at about 9 months after kidney treatment. In some aspects, the sample in the above method is obtained from the subject at about 12 months after kidney treatment.

After the sample is obtained, the level of at least one of IL-16, Galectin-1, CD163, CD206, FOLR2, and/or proteinase 3 and optionally, at least one biomarker from Table 1, is determined using one of the methods or techniques described previously herein. Once the level of at least one of these biomarkers is determined, it is compared to at least one control level for the same biomarker as also described previously herein. In some aspects, the control level for the same biomarker can be the level of the biomarker obtained from the subject prior to the start of treatment. In other aspects, the control level for the same biomarker can be the level of the biomarker obtained from the subject at one or more time points during or throughout the course of treatment. For example, the control level used for the comparison could be the level of the same biomarker(s) during the subject's previous appointment(s) with the treating clinician.

After completion of the comparison, a determination is made that the subject is responding to treatment for the lupus nephritis, such as, proliferative lupus nephritis, if the level of at least one of IL-16, Galectin-1, CD163, CD206, FOLR2, and/or proteinase 3 and optionally, at least one biomarker from Table 1, is lower than the control level for the same biomarker. A determination is made the subject is not responding to treatment for the lupus nephritis, such as, proliferative lupus nephritis, if the level of the at least one of IL-16, Galectin-1, CD163, CD206, FOLR2, and/or proteinase 3 and optionally, at least one biomarker from Table 1, is equal to or higher than the control level for the same biomarker. In the instances where the patient is not responding to treatment, the treating clinician may choose to increase the amount of treatment being administered or switch the subject to a new treatment. In some aspects, if the level of at least one of IL-16, Galectin-1, CD163, CD206, FOLR2, and/or proteinase 3 and optionally, at least one biomarker from Table 1 which is obtained after about 3 months of treatment shows a decline compared to the control level, it is determined that the subject is responding to treatment for the lupus nephritis such as proliferative lupus nephritis about 1 year after treatment. In other words, the decline of the level of at least one of IL-16, Galectin-1, CD163, CD206, FOLR2, and/or proteinase 3 and optionally, at least one biomarker from Table 1 which is obtained after about 3 months of treatment strongly predicts treatment response at about 1 year.

The method described herein can be repeated as often as needed to monitor the efficacy of a treatment in a subject suffering from lupus nephritis, such as, proliferative lupus nephritis.

The determination of the presence of and/or amount, level and/or concentration of IL-16, Galectin-1, CD163, CD206, FOLR2, and/or proteinase 3 and optionally, any biomarker listed in Table 1, pursuant to the methods described herein is not limited to any particular type of detection technique. For example, in some aspects, the at least one of IL-16, Galectin-1, CD163, CD206, FOLR2, and/or proteinase 3 can detected using fluorescent detection, spectrometric detection, chemiluminescent detection, matrix assisted laser desorption-time-of flight (MALDI-TOF) detection, high pressure liquid chromatographic detection, charge detection, mass detection, radio frequency detection, and light diffraction detection.

In some aspects, aptamers and/or next generation sequencing can be used to determine the presence of and/or amount, level and/or concentration IL-16, Galectin-1, CD163, CD206, FOLR2, and/or proteinase 3 and optionally, any biomarker listed in Table 1.

In other aspects, the at least one the biomarkers of IL-16, Galectin-1, CD163, CD206, FOLR2, proteinase 3, or any combination thereof is detected by performing or conducting an assay. The type of assay performed or conducted is not critical. In some aspects, the assay can employ or utilize one or more specific binding partners, wherein at least one of specific binding partners is used to capture (e.g., a capture molecule) at least one of the analyte of interest (a biomarker such as one or more of IL-16, Galectin-1, CD163, CD206, FOLR2, or proteinase 3 and optionally, any biomarker listed in Table 1). Examples of capture molecules include one or more antibodies that specifically bind to one or more of IL-16, Galectin-1, CD163, CD206, FOLR2, and/or proteinase 3. Optionally, or additionally, at least one second specific binding partner which also binds to the analyte of interest (a biomarker such as one or more of IL-16, Galectin-1, CD163, CD206, FOLR2, or proteinase 3) and which may be labeled with at least one detectable label, can also be used (e.g., a detection molecule). Examples of such assays which use such one or more specific binding partners (including capture and/or detection molecules) include: (1) an immunoassay, such as for example, an enzyme immunoassay (EIA), radioimmunoassay (RIA), fluoroimmunoassay (FIA), chemiluminescent immunoassay (CLIA), or counting immunoassay (CIA); (2) an enzyme-linked immunosorbent assay (ELISA), such as a direct ELISA, an indirect ELISA, a sandwich ELISA, or a completive ELISA; (3) agglutination assay; or (4) a complement fixation assay.

In other aspects, a protein microarray is used for detection. In this aspect, one or more specific binding partners (e.g., such as an antibody) are immobilized on a solid support, such as a biochip. A biological sample from a patient suspected of having lupus nephritis or having lupus nephritis is passed over the solid support. Bound IL-16, Galectin-1, CD163, CD206, FOLR2, and/or proteinase 3 and optionally, any biomarker listed in Table 1, are then detected using any technique known in the art.

In yet further aspects, the methods described herein comprise displaying the determination (e.g., detection of the presence of and/or the level, amount and/or concentration of at least one of IL-16, Galectin-1, CD163, CD206, FOLR2, and/or proteinase 3 in a sample) on at least one instrument. Suitable instruments include, a point-of-care device, a core laboratory device (e.g., such as an immunoassay analyzer), a clinical chemistry analyzer, a mass spectrometer, etc. that may contain a user interface that can display the determination.

In some embodiments, the instrument contains software to execute one or more tasks. In some embodiments, the instrument contains software to automatically determine the next appropriate step in a method as described herein. For example, the instrument may contain software that determines the presence of, whether levels are not elevated, and/or whether the test needs to be repeated. The software may display this determination, such as on a graphical user interface.

In some embodiments, the instrument stores software that instructs a processor to execute a given task. In some embodiments, the software stores machine readable instructions that instruct a processor to execute a given task. The machine readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

The machine readable instructions may be stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

In some embodiments, the present disclosure relates to a method for determining a type or grade of lupus nephritis in a subject, the method comprising the steps of:
(a) obtaining a biological sample from a subject being treated for lupus nephritis;
(b) determining a level at least one biomarker in the sample, wherein at least one of the biomarkers are IL-16, CD163, Catalase, PRTN3, S100A8, Azurocidin, and MMP8, or a combination thereof; and
(c) determining that the type or grade of the lupus nephritis is proliferative when the level of the at least one biomarker determined in step (b) is higher than the control level for the same biomarker or that the type or grade of the lupus nephritis is pure membranous when the level of the at least one biomarker determined in step (b) is less than the control level for the same biomarker.

In some aspect, the type of grade of lupus nephritis includes pure proliferative, mixed proliferative, and pure membranous LN. In one aspect, the proliferative LN signature is dominated by higher levels of a macrophage marker, such as CD163, a proinflammatory chemokine, such as IL-16, and neutrophil degranulation products, such as Catalase, PRTN3, S100A8, Azurocidin, and MMP8 or a combination thereof. In some aspects, the neutrophil degranulation, the macrophage activation, and the extracellular matrix degradation are implicated in LN activity.

### 3. Kits

Provided herein is a kit, which may be used for assaying or assessing a biological sample for at least one of IL-16, Galectin-1, CD163, CD206, FOLR2, and/or proteinase 3. The kit comprises reagents to detect the presence of or determine the level, amount and/or concentration of IL-16, Galectin-1, CD163, CD206, FOLR2, and/or proteinase 3 in the sample. In some embodiments, the kit can be used to detect or determine the level, amount and/or concentration of a panel of IL-16, Galectin-1, CD163, CD206, FOLR2, and proteinase 3.

The kit can contain at least one component (e.g., one or more antibodies) for detecting the presence of or determining the level, amount and/or concentration of IL-16, Galectin-1, CD163, CD206, FOLR2, and/or proteinase 3 and instructions for detecting the presence of or determining the level, amount or concentration in the test sample for IL-16, Galectin-1, CD163, CD206, FOLR2, and/or proteinase 3 in the sample. In some aspects, the one or more components may be immobilized or bound to a solid support, such as, for example, a biochip array, a microtiter plate, a stick or a bead (e.g., a microbead). Additionally, instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In one aspect, the at least one component may include at least one composition comprising one or more isolated antibodies or antibody fragments thereof that specifically bind to IL-16, Galectin-1, CD163, CD206, FOLR2, and/or proteinase 3. The antibody may be an anti-IL-16, anti-Galectin-1, anti-CD163 and/or anti-proteinase 3 capture antibody and/or an anti-IL-16, anti-Galectin-1, anti-CD163 and/or anti-proteinase 3 detection antibody.

Alternatively or additionally, the kit can comprise a calibrator or control, e.g., purified, and optionally lyophilized, IL-16, Galectin-1, CD163, CD206, FOLR2, and/or proteinase 3, and/or at least one container (e.g., tube, microtiter plates or strips, which can be already coated with an anti-IL-16, anti-Galectin-1, anti-CD163 and/or anti-proteinase 3 capture antibody monoclonal antibody(ies)) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying IL-16, Galectin-1, CD163, CD206, FOLR2, and/or proteinase 3. The reference standards may be employed to establish standard curves for interpolation and/or extrapolation of IL-16, Galectin-1, CD163, CD206, FOLR2, and/or proteinase 3 concentrations.

If antibodies are included in the kit, such as recombinant antibodies specific for IL-16, Galectin-1, CD163, CD206, FOLR2, and/or proteinase 3, they can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., IL-16, Galectin-1, CD163, CD206, FOLR2, and/or proteinase 3) or reagents for detecting the analyte (e.g., IL-16, Galectin-1, CD163, CD206, FOLR2, and/or proteinase 3). The antibodies, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the biological sample. The kit can also include one or more instruments for assisting with obtaining a test sample, such as a syringe, pipette, forceps, measured spoon, or the like.

The following examples have been included to provide guidance to one of ordinary skill in the art for practicing representative embodiments of the presently disclosed subject matter.

In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### EXAMPLES

### Example 1: Urine Proteomics and Renal Single Cell Transcriptomics Implicate IL-16 in Lupus Nephritis

### Methods

### Patients and Sample Collection

This study enrolled SLE patients with urine protein/creatinine ratio greater than 0.5 undergoing clinically indicated renal biopsy. Only patients with a pathology report confirming LN were included in the study. Renal biopsies were scored by one renal pathologist at each site of the two sites according to the International Society of Nephrology/Renal Pathology Society (ISN/RPS) guidelines and NIH activity and chronicity indices (15). Clinical information, including serologies, were collected at the most recent visit before the biopsy. Response status at week 52 was defined as follows. Complete: pr/cr≤0.5, normal serum creatinine (sCr) or <25% increase from baseline if abnormal, and prednisone ≤10 mg daily; partial: pr/cr>0.5 but ≤50% of the baseline value and identical sCr and prednisone rules as complete response; no response: pr/cr>50% of baseline value or new abnormal elevation of sCr or ≥25% from baseline or prednisone ≥10 mg daily. Urine samples from healthy volunteers (all females, median age 42 years [32-54], 3 identifying as Caucasian and 4 as African American) were included. Urine specimens were acquired on the day of the biopsy (before the procedure) at 2 clinical sites in the United (Johns Hopkins University, JHU, and New York University, NYU). For the validation cohort (n=101), urine samples were collected on the day of (73%) or within 3 weeks (27%) of kidney biopsy. Serological features and complement levels were determined at the clinical visit preceding the biopsy. Proteinuria was measured on or near the day of the biopsy.

### Study Approval

Human study protocols were approved by the institutional review boards at JHU and NYU, and written informed consent was received from all participants, including healthy controls.

### Urine Quantibody Assay

The Kiloplex Quantibody protein array platform (Raybiotech) was used for screening urine samples as previously described (13) and summarized in the Supplementary Methods.

### Renal Tissue Single Cell RNA Sequencing

Renal tissue was collected, stored and processed as previously described (16). Briefly, research biopsy cores were collected from consented subjects as an additional biopsy pass or tissue from routine clinical passes. Only biopsies with confirmed LN were included. Kidney tissue was frozen on site and shipped to a central processing location where it was thawed and disaggregated. Individual cells were retrieved and sorted by flow cytometry. For each sample, 10% of the sample was allocated to sort CD10+CD45− epithelial cells as single cells, and the remaining 90% of the sample was used to sort CD45+ leukocytes as single cells. For each single cell, the whole gene expression profile was sequenced using the CEL-Seq2 method.

### Pathway Enrichment Analysis

See Supplementary Methods.

### Differential Urine Protein Abundance

The differential protein abundance was calculated using a moderated t statistic (20). To achieve normal distribution, the protein abundances were log-transformed after adding 10% (arbitrary constant empirically shown not to significantly alter distributions) of the lowest measured abundance to remove zeros. With 30 LN and 7 HD samples, using a two-sided 0.05-level test adjusting for 1000 comparisons (Bonferroni), there was 80% power to detect a difference in mean peptide magnitude of 1.2 standard deviations (i.e., an effect size of 1.2).

### Receiver Operating Characteristic (ROC) Curves

ROC curves and areas under the curve (AUCs) were calculated using the function roc within the pROC R package (21).

### Regression Models

The impact of confounders on the association between the ISN Activity Index and the urinary abundance of a biomarker was tested using one confounder at the time (given limited sample size) using a linear regression model as follows: activity ˜biomarker_abundance+confounder. The models were fitted using the lm function within the stats R package (22).

### Prevalence of Cytokine Positive Cells

Analysis of cytokine-positive cells was based on a compendium of 237 cytokines obtained from Gene Ontology (19) and manually extended using the Cytokine Registry (https://www.immport.org/resources/cytokineRegistry), the iTalk database (23), and the International Union of Basic and Clinical Pharmacology (IUPHAR) and British Pharmacological Society (BPS) database (24). For each cytokine, the prevalence of the cells with at least one transcript over the total number of cells was calculated.

### Immunohistochemistry

See Supplementary Methods.

### Supplementary Methods

### Urine Quantibody Assay

The array was spotted with 1000 capture antibodies specific for 1000 different proteins in quadruplicate. The 1000 proteins printed on the arrays include the most common proteins in proteomic studies selected by the manufacturer. All urine samples were clarified by centrifugation, and then diluted to yield a total protein concentration within the working range (0.5-1 mg/mL) before application to the arrays as previously described (13). Samples were run in a single batch in random order to minimize batch potential batch effect. Briefly, protein standards and urine samples were incubated on the array for 2 hours to allow the proteins to bind to the capture antibodies. A biotinylated antibody cocktail comprised of 1000 detection antibodies was subsequently added for incubation for 2 hours. Finally, streptavidin-Cy3 was added and left to incubate for 1 hour. Washing was performed between each step to remove the unbound reagents. After a final wash and dry, the slides were read with a fluorescent scanner, and data were extracted from the image using vendor-provided GAL file with a suitable microarray analysis software. Creatinine was measured for each urine sample (KGE005, R&D Systems, Inc., Minneapolis, MN). Normalization was performed as previously described (13). Briefly, intraassay normalization was performed using positive control spots and standard curves across multiple slides as directed by the manufacturer. All data were normalized by urine creatinine (mg/dl) before analysis to reduce the potential bias from differentially concentrated urine samples between patients. The calibration studies showed excellent signal to concentration curves with median R² of 0.99 (IQR 0.99-1). The validity of Kiloplex to quantify soluble biomarkers was previously confirmed by ELISA (13). This assay was selected to measure the broadest possible array of urinary proteins while maintaining high sensitivity. For an unbiased approach, no specific protein or pathway was excluded or selected.

### Human IL-16 IQELISA.

Previously frozen (−80 C) urine samples that were used for the external validation cohort were thawed and diluted 1:3 before soluble IL-16 was quantified in triplicates using the PCR-based Human IL-16 IQELISA Kit (Raybiotech, IQH-IL16-2) according to the manufacturer instructions.

### Pathway Enrichment Analysis.

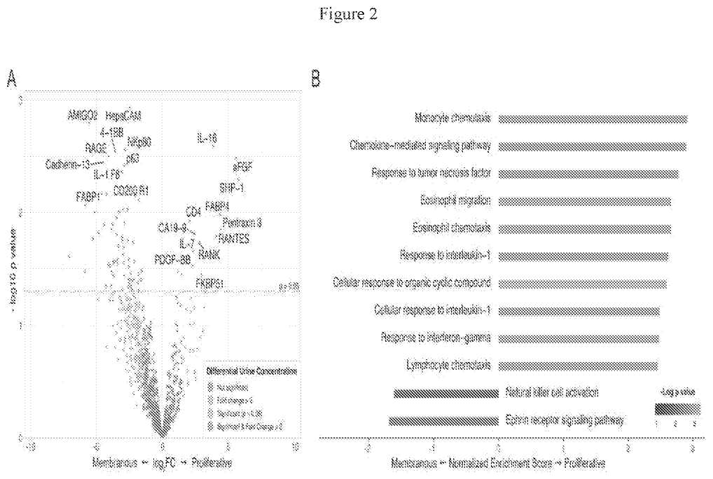

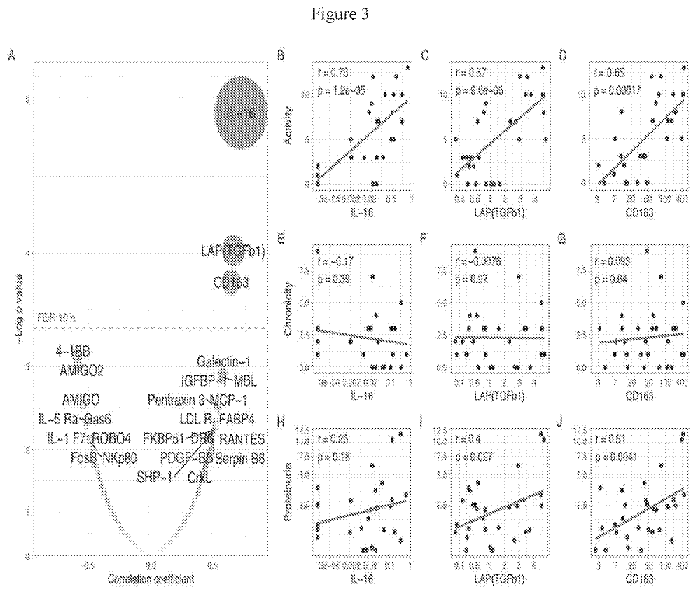

Pathway enrichment analysis was performed using protein-coding genes. Because a limited gene/protein universe of 1000 features was probed by the protein array, self-contained enrichment approaches (17) such as the Fisher exact test (hypergeometric distribution) or gene set enrichment analysis (GSEA) were employed (18). The Fisher exact test was used for discrete sets of differentially abundant proteins/genes identified by comparing 2 groups (i.e., FIG. 2). GSEA was used for the analysis of the correlation of the 1000 features with continuous variables such as the NIH activity index (i.e., FIG. 3A) ranked by the correlation coefficient. Only Gene Ontology (GO) terms (19) with at least 10 genes included by the Quantibody array were retained. To limit redundant information from overlapping gene sets, only pathways with Szymkiewicz-Simpson overlap coefficient <0.5 were retained as non-overlapping.

### Immunohistochemistry.

Formalin fixed and paraffin embedded kidney tissue slides were deparaffinized with xylene and rehydrated with gradient concentrations of ethanol. For antigen retrieval, the slides were transferred into a pressure cooker including a citrate solution (BioSB Inc, Catalog No. BSB 0022), boiled with high pressure for 15 min then slowly cooled down to room temperature. After retrieval, slides were subjected to the procedure of immunohistochemistry. Briefly, slides were blocked with a peroxidase blocker (Bio SB Catalog No. BSB 0054) for 5 minutes, washed with an immunoDNA Washer buffer (Bio SB, Catalog No. BSB 0150) once, continually blocked with an antibody diluent (Bio SB Catalog No. BSB 0041) for 20 minutes. Then, the slides were incubated with 1:1000 of rabbit anti-human IL16 antibody (ATAS ANTIBODIS, catalog No. HPA018467) for 1 hour. After three washes, a high sensitivity Mouse/Rabbit PolyDetector Plus DAB HRP Brown Detection System (Bio SB, Catalog No. BSB 0269) was used to develop tissue staining following manufacture's instruction. Hematoxylin used for counterstain.

### Results

### Urine Proteomics Identifies Biologically Relevant Active Pathways in LN

Urine samples from 30 subjects with active LN were collected near or at the time of a renal biopsy. Clinical and demographic characteristics are summarized in Table A.

TABLE A
Clinical and demographic characteristics

| | n (%)/mean | |
|---|---|---|
| Characteristic | (range) | |
| Female | 28 | (93%) |
| Age | 35 | (19-54) |
| Race/Ethnicity | | |
| Black/African American | 12 | (40%) |
| White | 10 | (33%) |
| Asian | 7 | (23%) |
| Other | 1 | (3%) |
| Hispanic | 7 | (23%) |
| Anti-dsDNA | 24 | (80%) |
| Low C3 | 24 | (80%) |
| Low C4 | 18 | (60%) |
| Lupus nephritis class | | |
| III or IV | 14 | (47%) |
| V | 9 | (30%) |
| III + V or IV + V | 7 | (23%) |
| Histological features (n = 26) | | |
| NIH Activity Index | 5 | (0-13) |
| NIH Chronicity Index | 1 | (0-9) |
| Proteinuria, mg/g | 2.6 | (0.53-11.6) |
| Proteinuria >3,000 mg/g | 7 | (23%) |
| Serum creatinine | 1.0 | (0.5-5.2) |
| Abnormal serum creatinine | 3 | (10%) |
| Response to treatment at 12 months | | |
| Complete | 5 | (18%) |
| Partial | 5 | (18%) |
| None | 18 | (64%) |
| Treatment at time of biopsy | | |
| Hydroxychloroquine | 26 | (87%) |
| Prednisone | 19 | (63%) |
| Mycophenolate | 5 | (17%) |
| Belimumab | 2 | (7%) |
| ACE inhibitor/angiotensin receptor | | |
| blocker | 5 | (17%) |

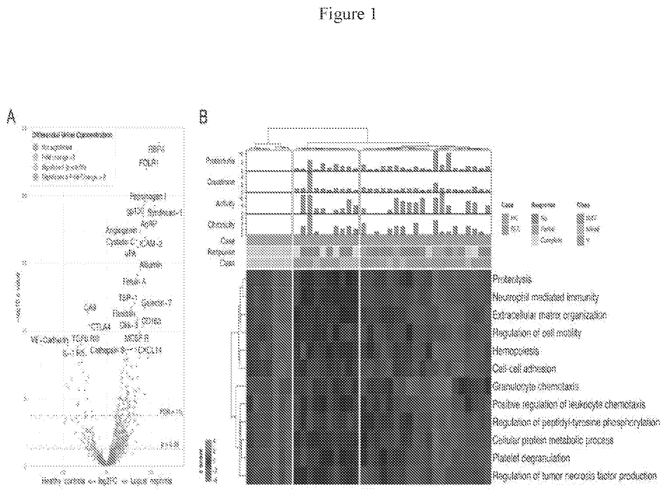

Compared to healthy donors, there were 237 proteins significantly elevated in the urine of patients with LN (FDR <10%) as displayed in FIG. 1A. This list included both new and previously described urinary. Pathway enrichment analysis of the proteins the proteins significantly elevated in LN identified 12 enriched non-overlapping pathways, including relevant biological processes such as chemotaxis, neutrophil activation, platelet degranulation, and extracellular matrix organization (FIG. 7). Hierarchical clustering using enriched pathways segregated LN patients into 2 groups, with 80% of those who later achieved a complete renal response being in the same group with overall less inflammatory pathways (OR 12.6, p=0.03) (FIG. 1B). Baseline parameters such as proteinuria, creatinine, histologic activity or chronicity scores, or class were present in similar frequencies in both clusters suggesting that urine proteomics may provide unique informative features (FIG. 1B).

### Identification of Biomarkers to Predict Renal Histology.

It was determined to identify urinary proteins that could predict renal histology. LN can be classified in two broad categories based on the presence of a glomerular endocapillary immune infiltrate or “proliferation”. Proliferative LN (ISN class III or IV) is a more aggressive phenotype associated with glomerular endocapillary hypercellularity, abundant immune cell infiltration and higher risk of permanent renal damage (25). Compared to pure membranous LN (n=9), patients with proliferative LN (n=14) showed higher concentration of several urine cytokines and molecules involved in immune activation and chemotaxis (FIG. 2A-B). IL-16 was the most significantly enriched urinary protein in proliferative LN (FIG. 2A). Pathway enrichment analysis revealed that the pattern of chemokines matched the chemokine released in response to interferon-gamma (IFN-γ), IL-1β, and TNF (FIG. 2B).

Many of the urinary proteins that were differentially abundant when comparing proliferative and membranous were not significantly more abundant when comparing all LN patients to healthy controls. In fact, although most of the proteins enriched in proliferative LN were generally more abundant in LN vs healthy controls, these were not among the most abundant (>2 SD) (FIG. 8 A-B). This is because the first comparison (LN vs healthy) is aimed to identify proteins that are generally more abundant in all LN patients, regardless of class. Not surprisingly, the most abundant protein in all LN was RBP4, a general marker of tubular impairment (26). These findings indicate that contrasting well defined subgroups allowed to identify relevant biomarkers that could have been missed by analyzing all LN patients together. Different pathogenic processes may underlie each histological subgroup and thus these biomarkers may provide insight into the relative active pathways.

### Urinary IL-16 Predicts Histological Activity.

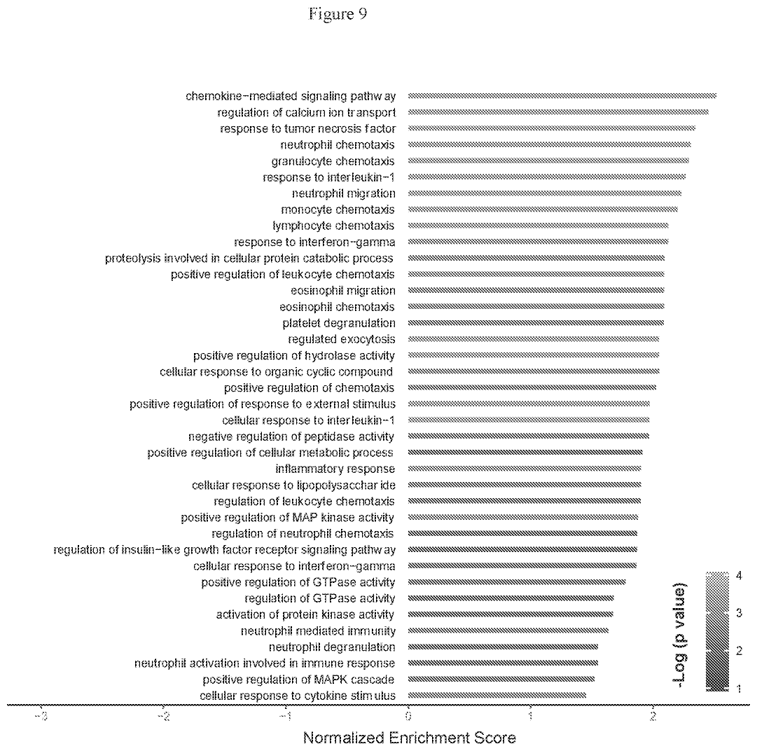

The degree of histological activity is often used to inform clinical decisions, so it was sought to identify noninvasive urinary biomarkers that could predict activity. The correlation of the urinary abundances of all 1000 biomarkers in urine samples collected at the time of biopsy with the histological NIH activity index was studied. It was found that IL-16 was the urinary protein most strongly positively correlated with the NIH activity index (Pearson's r 0.73, p=1.2*10⁻⁵, FDR <10%, n=28) followed by CD163, and TGF-β (FDR <10%) (FIG. 3A-D). The significant correlation between urinary IL-16 abundance and NIH activity index in an independent cohort of 101 patients was validated (r=0.59, p=9.3*10⁻¹¹, FIG. 9 and Table B).

TABLE B
The association between urinary biomarkers and
histological activity is independent of confounders.

| | IL-16 | | CD163 | | TGF-β1 | |
|---|---|---|---|---|---|---|
| Confounder | Estimate | p | Estimate | P | Estimate | P |
| Unadjusted | 0.79 | 1.10E−05 | 1.3 | 1.70E−04 | 2 | 9.58E−05 |
| Age | 0.77 | 1.70E−05 | 1.2 | 5.10E−04 | 2 | 4.10E−04 |
| Race | 0.78 | 1.00E−04 | 1.2 | 1.20E−03 | 2.1 | 5.50E−04 |
| U pr/cr | 0.77 | 4.70E−05 | 1.3 | 7.90E−04 | 2.1 | 4.50E−04 |
| Anti-dsDNA | 0.77 | 6.00E−06 | 1.3 | 1.70E−04 | 2 | 6.40E−05 |
| C3 | 0.78 | 1.30E−05 | 1.3 | 4.10E−04 | 2 | 1.80E−04 |
| C4 | 0.8 | 4.00E−05 | 1.3 | 3.20E−04 | 2 | 2.70E−04 |
| Creatinine | 0.75 | 8.80E−06 | 1.2 | 4.40E−04 | 1.9 | 2.40E−04 |
| Prednisone | 0.79 | 1.60E−05 | 1.3 | 2.30E−04 | 2 | 1.30E−04 |
| MMF | 0.81 | 1.30E−05 | 1.4 | 1.60E−04 | 2.1 | 1.00E−04 |
| Site | 0.81 | 2.40E−05 | 1.3 | 3.50E−04 | 2.1 | 2.00E−04 |

Notably, IL-16 was the only one not associated with proteinuria (FIG. 3H), suggesting the potential to provide actionable information in addition to classic biomarkers such as proteinuria. In multivariate models, IL-16, CD163, and TGF-β retained their association with histological activity after adjustment for multiple confounders, including proteinuria (Table C).

TABLE C
The association between urinary biomarkers and
histological activity is independent of confounders

| | IL-16 | | CD163 | | TGF-β1 | |
|---|---|---|---|---|---|---|
| Confounder | Estimate | p | Estimate | p | Estimate | p |
| Unadjusted | 0.79 | 1.10E−05 | 1.3 | 1.70E−04 | 2 | 9.58E−05 |
| Age | 0.77 | 1.70E−05 | 1.2 | 5.10E−04 | 2 | 4.10E−04 |
| Race | 0.78 | 1.00E−04 | 1.2 | 1.20E−03 | 2.1 | 5.50E−04 |
| U pr/cr | 0.77 | 4.70E−05 | 1.3 | 7.90E−04 | 2.1 | 4.50E−04 |
| Anti-dsDNA | 0.77 | 6.00E−06 | 1.3 | 1.70E−04 | 2 | 6.40E−05 |
| C3 | 0.78 | 1.30E−05 | 1.3 | 4.10E−04 | 2 | 1.80E−04 |
| C4 | 0.8 | 4.00E−05 | 1.3 | 3.20E−04 | 2 | 2.70E−04 |
| Creatinine | 0.75 | 8.80E−06 | 1.2 | 4.40E−04 | 1.9 | 2.40E−04 |
| Prednisone | 0.79 | 1.60E−05 | 1.3 | 2.30E−04 | 2 | 1.30E−04 |
| MMF | 0.81 | 1.30E−05 | 1.4 | 1.60E−04 | 2.1 | 1.00E−04 |
| Site | 0.81 | 2.40E−05 | 1.3 | 3.50E−04 | 2.1 | 2.00E−04 |
| IL-16 | | | 0.6 | 1.00E−01 | 0.88 | 1.50E−01 |
| CD163 | 0.58 | 5.60E−03 | | | 1.3 | 1.00E−01 |
| TGF-β1 | 0.56 | 1.40E−02 | 0.65 | 2.00E−01 | | |

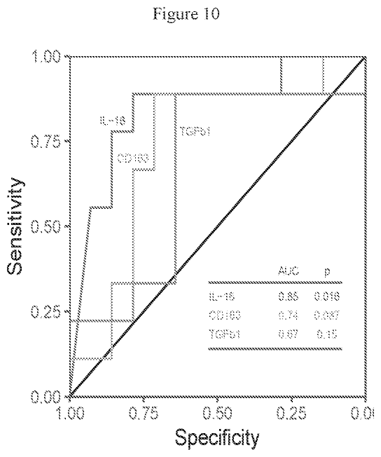

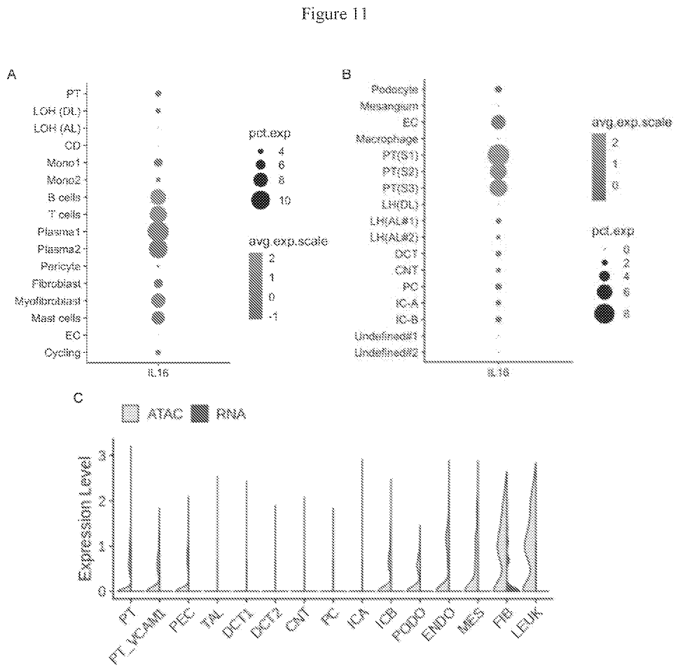

The pathways associated with histological activity are displayed in FIG. 10. In addition to having the strongest correlation with histological activity, IL-16 was the urinary protein most associated with proliferative LN (FIG. 2A). The receiver operating characteristic curve revealed that IL-16 was a promising urinary biomarker to identify patients with proliferative LN with an area under the curve (AUC) of 0.85 (p=0.016) and 0.89 (p=0.037) in association with CD163 and TGF-β (FIG. 11).

Urinary Biomarkers Correlating with Activity Decrease with Clinical Response in Longitudinal Samples.

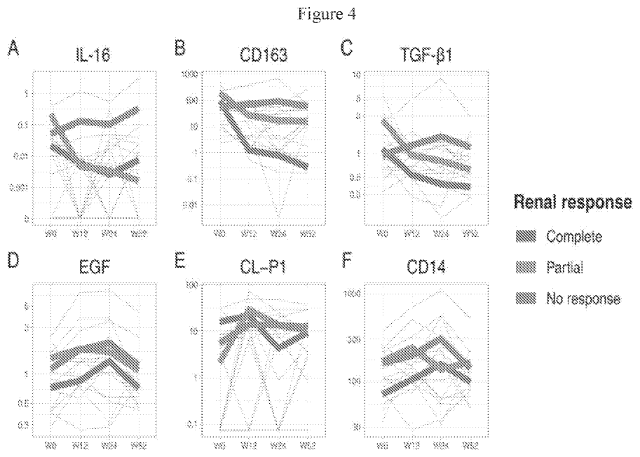

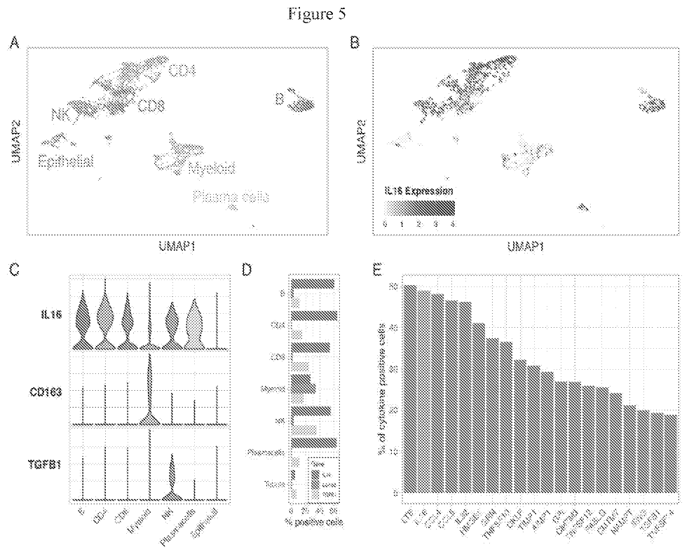

A goal of immunosuppression in LN is to eradicate pathological renal inflammation to ultimately prevent irreversible renal damage and preserve function. The NIH activity index captures many renal inflammatory features and, as a consequence, it improves with treatment in patients achieving renal remission (3, 27). However, it is impractical to monitor in clinical practice as it requires frequent repeat renal biopsies. Thus, it was hypothesized that the 3 urinary biomarkers associated with histological activity would decline over time in patients who are responding to treatment and might serve as noninvasive biomarkers of response. The urinary concentration of all 3 candidate biomarkers declined in complete and partial responders but not in non-responders (FIG. 4A-C). The average decline was most striking in IL-16 with a decrease in partial and complete responders by week 12. CD163 concentration improved by week 12 in complete responders but not in partial responders. TGF-β showed a more modest decline.

Since response status is defined by reduction of proteinuria, it was decided to ensure that the observed biomarker trajectories were not simply a reflection of a decline in all urinary protein in responders. The trajectories of 3 urinary proteins that were selected among those that did not correlate with histological activity demonstrated that there was no non-specific decline (FIG. 4D-F). These findings indicate that IL-16, CD163, and TGF-β trajectories represent a specific decrease in the production and excretion of these molecules and, as they strongly correlated with activity at baseline, likely reflect a corresponding improvement of intrarenal LN activity supporting their value as biomarkers.

### IL-16 is One the Most Expressed Cytokines in Kidney Infiltrating Immune Cells in LN.

To determine whether the urinary concentration of the 3 candidate biomarkers reflects an active intrarenal process rather than passive filtration through a damaged glomerular membrane, the intrarenal relative gene expression using single cell RNA sequencing of LN renal biopsies was evaluated. IL-16 was abundantly expressed by most immune infiltrating cells, CD163 by a subset of myeloid cells, and TGFB1 mostly by NK cells (FIG. 5A-D).

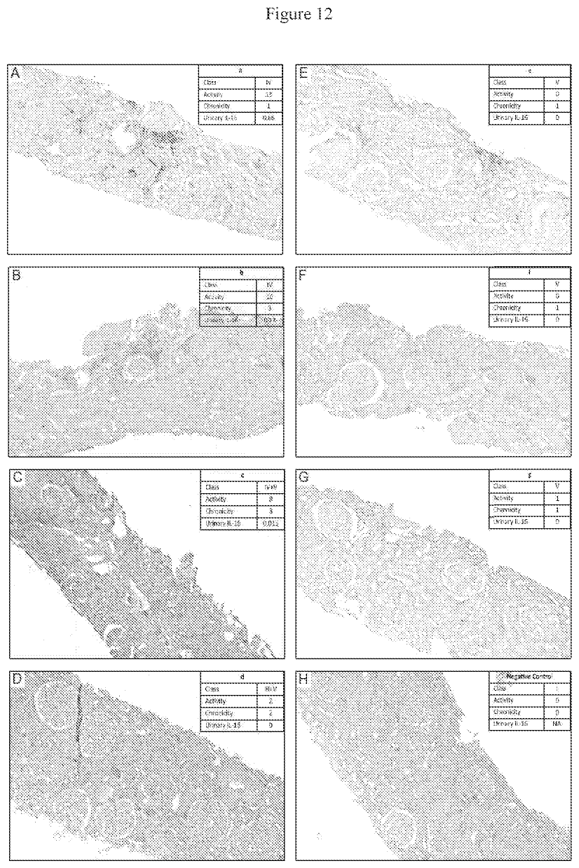

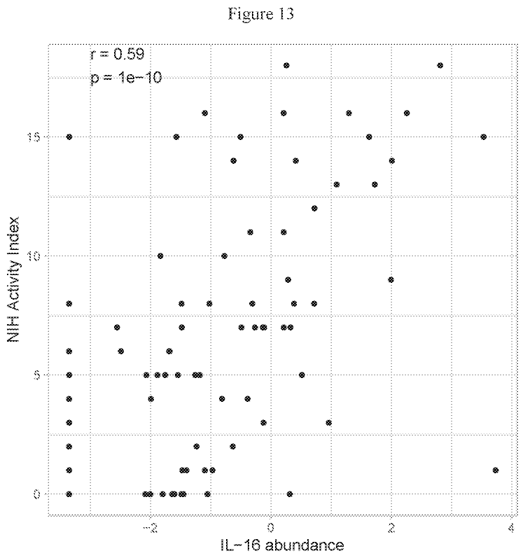

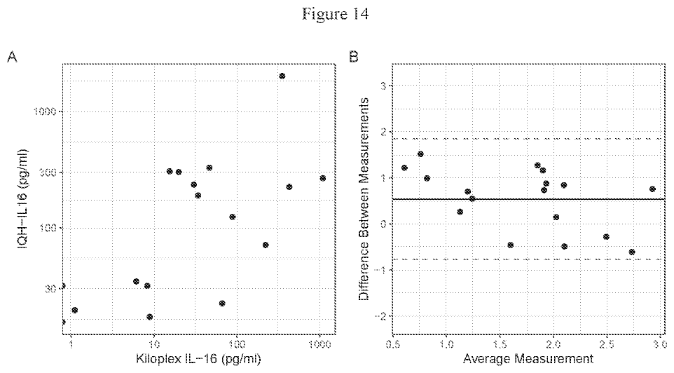

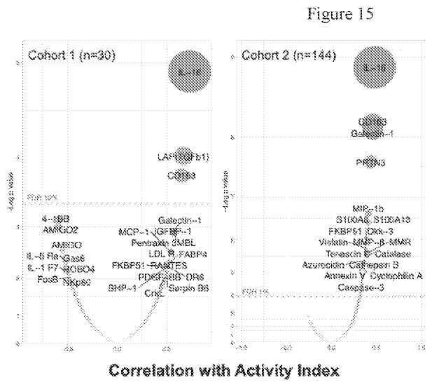

In LN, most of IL-16 expression was in immune infiltrating cells, especially the lymphoid lineage (FIG. 5C-D). In renal allograft rejection, single cell RNA-seq showed that IL-16 is expressed by endothelial, epithelial, and immune cells, but immune cells were the main source (FIG. 12A) (28). Conversely, in the healthy kidney, single nuclear RNA sequencing and ATAC-seq (Assay for Transposase-Accessible Chromatin using sequencing) revealed substantial IL-16 expression by podocytes, fibroblasts, endothelial, mesangial, and proximal tubular cells (FIG. 11B-C) (29, 30). These findings suggest that while immune cells are likely the major intrarenal source of IL-16 in LN, IL-16 secretion by endothelial and tubular cells may precede immune infiltration. Speculating, this initial event can then be amplified by infiltrating immune cells as seen in LN and allograft rejection.

Finally, it was explored whether IL-16 was disproportionally more expressed as compared to other cytokines in LN. Out of a compendium of 237 cytokines, IL-16 was the second most commonly expressed cytokine (49% of all infiltrating immune cells) (FIG. 5E). These findings independently nominate IL-16 as a major cytokine involved in LN.

Tissue Expression of IL-16 Correlate with LN Activity and Urinary IL-16 Abundance.

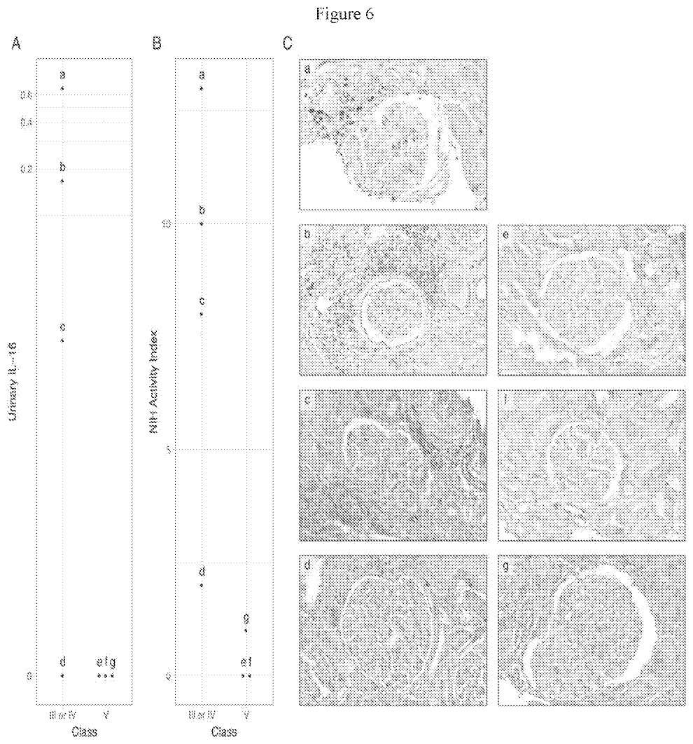

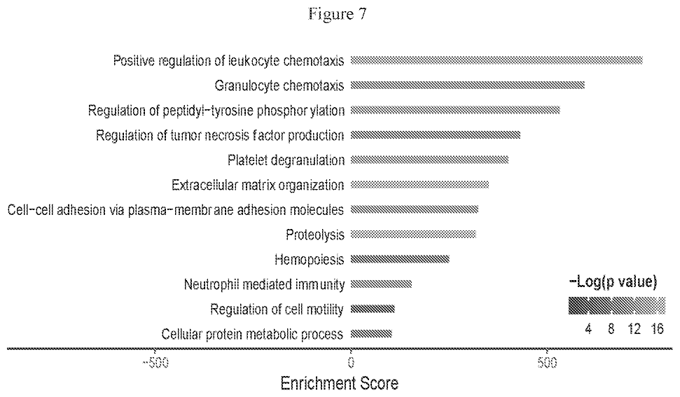

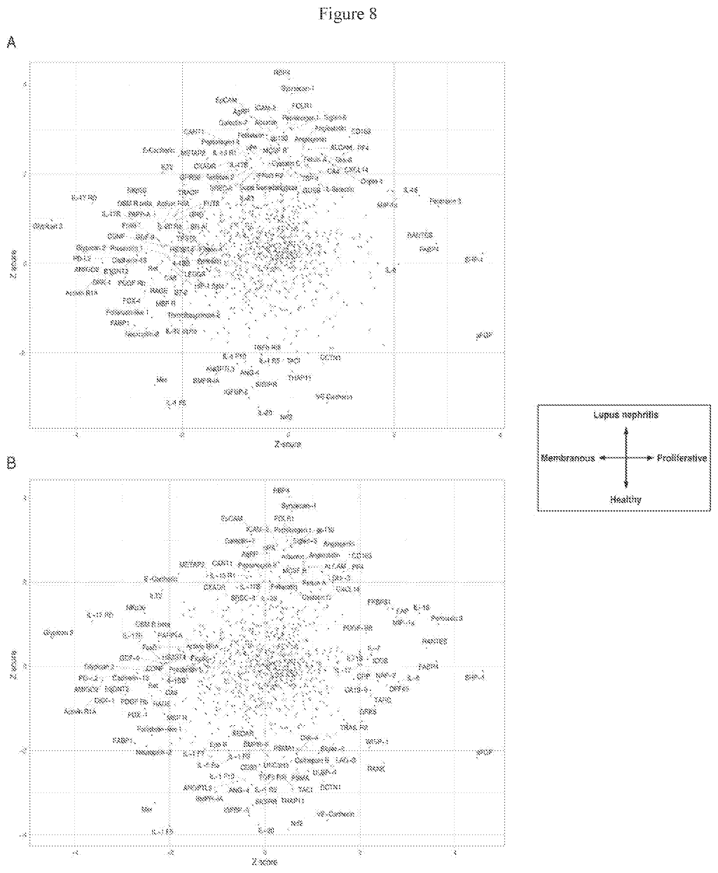

To establish the location of IL-16 secreting cells in renal tissue, immunohistochemical staining of human IL-16 in 7 LN kidney biopsies with matching urine IL-16 collected at or near the time of biopsy was performed. It was observed abundant interstitial and glomerular IL-16 expression in proliferative LN (FIGS. 6 and 12), with the exception of one case (FIG. 12D) in which the activity index was uncharacteristically low (2) and IL-16 was not detectable in the urine. In contrast, there was very scant IL-16 positivity in membranous LN (FIGS. 6 and 12E-G) and marginal in a class I LN biopsy used as negative control (FIG. 12H). These findings matched the urinary IL-16 profile. Furthermore, there was a qualitative correlation between the number of IL-16 positive cells and urinary IL-16 abundance as well as the NIH activity index (FIG. 6). This was particularly evident for glomerular IL-16 positive cells. These findings indicate that IL-16 is intrarenally produced in proliferative LN and urinary IL-16 reflects the abundance of intrarenal IL-16 positive cells and LN activity.

Leveraging urine proteomics in patients with LN and healthy controls, the results of this study confirmed that the pathological processes in LN can be noninvasively captured and monitored over time. It was found that: (1) 237 urinary proteins associated with LN that represented at least 12 distinct molecular pathways; (2) a strong chemokine signature characterizing the urine of patients with proliferative LN; and (3) several candidate biomarkers to predict active nephritis that can be monitored over time to assess response to treatment. Overall IL-16 emerged as the most robust correlate of histological activity implying a role in LN pathogenesis and thus subsequent translation to clinical application both as a biomarker and treatable target.

Proteomic analysis revealed that the intrarenal activation of several pathogenic mechanisms contributing to LN can be quantified in the urine. These biological processes were previously implicated in LN including neutrophil immunity (31, 32), platelet degranulation (33), extracellular matrix organization (34), and chemotaxis (35). Patients did not cluster based on the abundance of a single or a group of signatures. Rather, two clusters characterized by high and intermediate abundance of all signatures, respectively were observed. This is consistent with previous findings from an agnostic approach to urine proteomics in LN that showed that patients stratify on a gradient (35). Importantly, 80% of complete responders clustered in the intermediate abundance group. The predictive value of this approach needs to be validated in a larger cohort given the small number of responders.

In this study, urinary abundance of proteomic signatures was independent from proteinuria, suggesting that these signatures specifically reflect active biological processes rather than a non-specific increase or decrease of all urine proteins. In particular, pathway enrichment analysis revealed a strong chemokine signature in proliferative LN suggesting active recruiting of immune cells in the kidney in these patients. This is biologically consistent with the abundant immune cell infiltration and more aggressive phenotype observed in class III and IV LN, further supporting the ability of urine proteomics to infer intrarenal biological processes.

Ideal biomarkers in LN should noninvasively infer nephritis activity, longitudinally track response to treatment, and ideally capture the intrarenal biology. Based on feasibility, the current management of LN hinges on monitoring proteinuria to establish renal activity rather than frequent biopsies. However, proteinuria is a poor marker of nephritis activity. Six-month repeat biopsies after induction therapy revealed that about half of the patients in complete clinical remission (proteinuria <0.5 g/24 hours and no increase in sCr) had persistent histologically active proliferative nephritis (36). Conversely, >50% of patients who achieved complete histological remission had persistent proteinuria >0.5 g/24 hours.

Moreover, patients in clinical remission 3 years after induction treatment may show persistent nephritis activity on per protocol biopsies which is associated with flares of nephritis as immunosuppression is tapered (3). Using an unbiased approach, it was discovered a previously unrecognized biomarker of intrarenal activity, IL-16, in addition to two previously recognized LN biomarkers, CD163 (37) and TGF-β (38). IL-16 showed the strongest and most significant association with the renal activity index of any marker measured, and urinary abundance of IL-16 decreased over time in patients who ultimately responded to treatment after 1 year. IL-16, CD163, and TGF-β were selected based on their correlation with histological activity; thus, it is conceivable that their decreasing urinary abundance mirrored an improvement of intrarenal histological activity. In fact, urinary proteins that did not correlate with activity did not decrease over time in responders.

Renal single cell RNA sequencing revealed that IL-16, CD163, and TGFB1 are actively expressed by immune infiltrating cells in LN kidney biopsies, suggesting that their detection in the urine reflects intrarenal immune activity. Because their expression was in distinct immune cell types, their urinary abundance could identify the activity of distinct immune processes. It was discovered that IL-16 was the second most expressed cytokine in LN kidneys (49% of all infiltrating immune cells). This striking concordant result was independent of the urine proteomics dataset, thus demonstrating the relevance of IL-16 in LN in an orthogonal approach. Furthermore, it was demonstrated prominent intraglomerular and interstitial renal production of IL-16 in proliferative LN by immunohistochemistry. Although circulating cells or serum were not evaluated, IL-16 urinary abundance correlated with intrarenal IL-16 positive cells implicating that urinary IL-16 is the direct consequence of intrarenal IL-16 secretion. Because urinary IL-16, intrarenal IL-16 positive cells, and histological activity are positively co-correlated and IL-16 is one the most expressed cytokines in LN, these findings suggest that IL-16 may be implicated in LN pathogenesis and this process can be non-invasively measured in urine.

IL-16 is a proinflammatory chemokine secreted by immune cells and non-immune cells (endothelial, epithelial cells, fibroblasts, and neurons) in response to several stimuli such as complement activation, antigen stimulation, interferon, hypoxia, and cell injury (39-44). Because the release of bioactive IL-16 depends on caspase 3 activation (43), apoptosis and pro-apoptotic stimuli including sublethal doses of granzymes may also lead to its release. IL-16 can also be released upon cleavage by proteinase 3 (45) suggesting that urinary IL-16 may indicate neutrophil degranulation. IL-16 is the natural ligand for CD4 and CD9 and is a strong chemoattractant for CD4+ T cells (especially Th1 cells) as well as CD8 T, NK, B cells monocytes, neutrophils, dendritic cells, and mast cells (39). IL-16 can activate CD4 T cells independently of T-cell receptor (TCR) activation (46) and may lead to the release of proinflammatory cytokines such as TNF, IL-1β, IL-6, IL-15, and IL-12 (39). IL-16 polymorphisms were associated with increased risk of SLE (OR 3.3-10.4) suggesting a potential causal role (47). Plasma IL-16 levels were associated with SLE severity including renal involvement (48). Finally, IL-16 was mechanistically linked to lung disease in the pristane model of SLE (49). The role of IL-16 in LN is yet to be fully understood, but it has been implicated in several other immune mediated diseases such as multiple sclerosis, scleroderma, rheumatoid arthritis, and allograft rejection (39, 50, 51).

This study demonstrated the power of integrating urinary proteomic screening platforms with matching clinical and pathological information and with tissue single cell transcriptomics (52). In fact, in addition to a newly discovered biomarker, CD163 and TGF-β that are proven biomarkers in LN are detected. Similar to previous findings, soluble CD163 was shown to correlate with LN nephritis activity and improve with treatment (37). CD163 is a scavenger receptor expressed on phagocytic monocytes, especially in M2c polarized macrophages that infiltrate tissues during the healing phase of inflammation and are implicated in fibrosis resolution (53). Notably, M2c macrophage are inducible by TGF-β (54). CD163+ cells are a dominant macrophage subtype in LN (54), thus again supporting the capability of urinary proteomic to infer intrarenal biology. CD163+ cells have been detected in proliferative glomerular lesions and in tubulointerstitial inflammation (55, 56) and they constitute ˜80% of the urinary cells in LN (57). Similarly consistent with these results, urinary TGF-β correlated with nephritis activity and response in previous studies (38, 58, 59), but sensitive immunoassays (such as the one used here) are required to reliably detect urinary TGF-β (59). TGF-β regulates inflammation and progression of renal fibrosis. Notably, TGF-β increased IL-16 release in synovial fibroblasts suggesting a possible similar interplay between these two cytokines in LN (60). Here, it was shown that NK cells are the major immune cell type expressing TGFB1 in LN, whether NK or tubular cells (61) are responsible for urinary TGF-β in LN is to be determined.

In summary, this study linked IL-16 release with lupus nephritis activity suggesting a possible role as a biomarker and in LN pathogenesis thus nominating IL-16 as a potentially treatable target. Further, this study demonstrated the feasibility to detect new and biologically relevant biomarkers in LN using a urine proteomic platform in a well characterized longitudinal cohort.

### Example 2: IL-16 is Linked to Lupus Nephritis Activity

### Methods

Urinary proteins (up to 1200) were quantified (RayBiotech Kiloplex) in urine samples from two independent cohorts of SLE patients with lupus nephritis (n=30 and n=144). Samples were collected on the day of (73%) or within 3 weeks (27%) of kidney biopsy in SLE patients with proteinuria >500 mg/d. The NIH Activity Index was determined by a renal pathologist at each site. Intrarenal expression of candidate biomarkers was evaluated using single cell transcriptomics of renal biopsies from patients with active lupus nephritis (n=24).

### Results

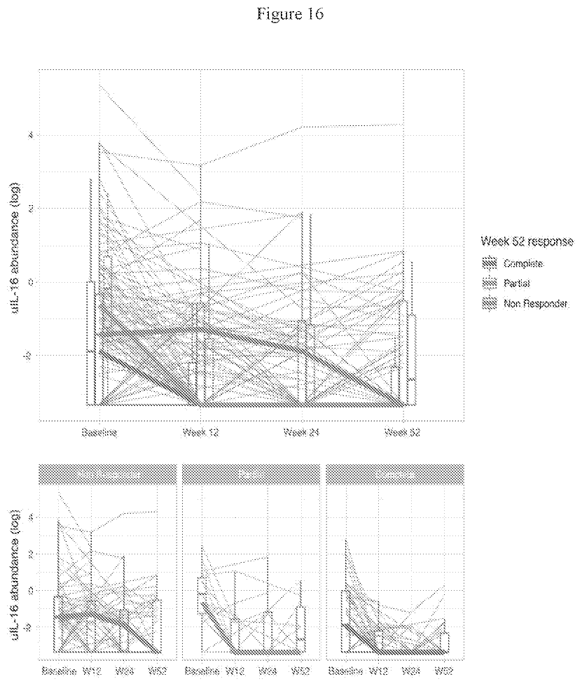

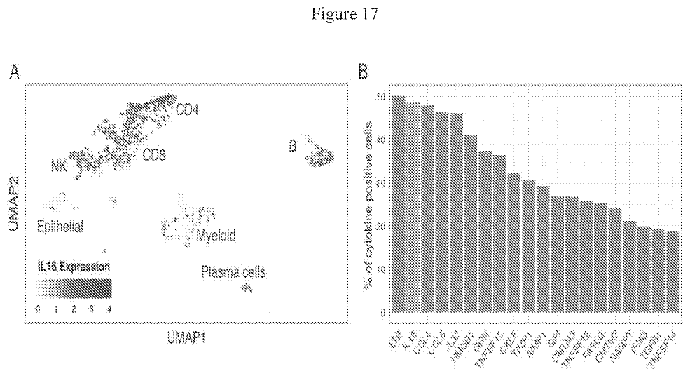

A total of 174 patients were included: 127 (73%) had a proliferative histological class (III or IV+/−V), 47 (27%) pure membranous (V). Urinary IL-16 showed the strongest positive correlation with histological activity (NIH Activity Index) in two independent cohorts (r=0.69, p=9·10⁻⁵; r=0.49, p=3·10⁻¹⁰; FIG. 15). A comprehensive list of proteins significantly correlated with histological activity is summarized by Table 1. Response to treatment was paralleled by an early reduction of urinary IL-16 (FIG. 16). Single cell RNA sequencing independently demonstrated that IL-16 is the second most widely expressed cytokine by most infiltrating immune cells in lupus nephritis kidneys (FIG. 17).

### Conclusions

Urine proteomics can profoundly change the diagnosis and management of lupus nephritis by noninvasively monitor active intrarenal biological pathways. These findings implicate IL-16, a proinflammatory chemokine, in lupus nephritis pathogenesis designating it as a potentially treatable target and biomarker.

### Example 3: A Neutrophil Degranulation Signature Identifies Proliferative Lupus Nephritis

### Background and Aims

The identification of intrarenal pathological processes is key to develop better diagnostic and treatment strategies in lupus nephritis (LN). But the direct comprehensive study of renal tissue can be limited by tissue degradation, availability, and cell survival. Therefore, urine proteomics were employed to define the molecular pathways involved in proliferative LN.

1200 biomarkers were quantified (Kiloplex, RayBiotech) in urine samples collected on the day of (73%) or within 3 weeks (27%) of kidney biopsy in SLE patients with urine protein to creatinine ratio on random or 24-hour collection of >0.5. Urine proteomic profiles were analyzed with respect to lupus nephritis histological features.

### Results

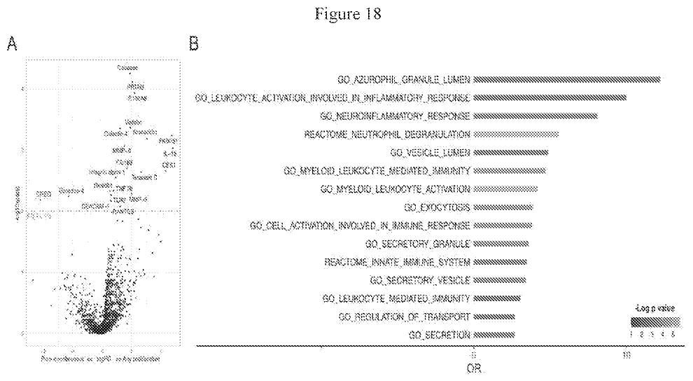

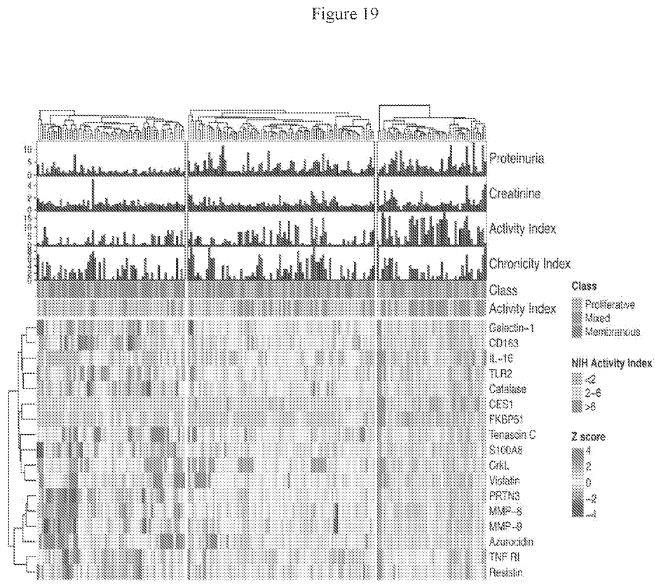

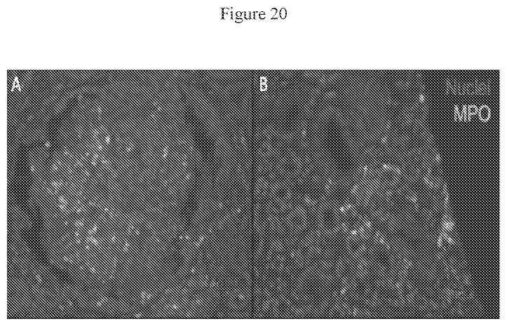

A total of 195 patients were included: 138 (71%) had a proliferative histological class (III or IV+/−V), 57 (29%) pure membranous (V). There were 21 (FDR 1%) differentially abundant urinary proteins in proliferative compared to pure membranous LN (FIG. 18A). These included several neutrophil granule proteins (FIG. 18B) in addition to previously reported biomarkers such as IL-16 and CD163. Unsupervised clustering based on the proliferative LN signature identified 3 groups characterized by low, medium or high protein abundance (FIG. 19). Higher proliferative signature abundance (right cluster) was associated with higher histological activity (NIH Activity Index). Immunofluorescence revealed an abundant MPO+ neutrophil infiltrate in proliferative LN (FIG. 20).

### Conclusions

Proliferative LN was associated with a urinary neutrophil degranulation signature, especially in patients with higher histological activity. Neutrophil activity could be non-invasively monitored to assist with the diagnosis of proliferative LN. These findings implicate neutrophils in LN activity and pathogenesis, nominate urinary neutrophil signatures as noninvasive biomarkers, and support the study of treatment targeted to neutrophils.

### Example 4: Urine Proteomic Signatures of Histological Class, Activity, Chronicity, and Treatment Response in Lupus Nephritis

### Methods

### Patients and Samples Collection

This study enrolled SLE patients with a urine protein-to-creatinine ratio (UPCR) of >0.5 who were undergoing clinically indicated renal biopsy. Only patients with a pathology report confirming LN were included in the study. Renal biopsy sections were scored by a renal pathologist at each site according to the International Society of Nephrology (ISN)/Renal Pathology Society guidelines and the National Institutes of Health (NIH) activity and chronicity indices (15). Clinical information, including serologies, were collected at the most recent visit before the biopsy. Response status at week 52 was defined in patients with a baseline UPCR >1 as follows: complete response (UPCR ≤0.5, normal serum creatinine or <25% increase from baseline if abnormal, and prednisone ≤10 mg daily), partial response (UPCR >0.5 but ≤50% of baseline value, and identical serum creatinine and pred-nisone rules as complete response), or no response (UPCR >50% of baseline value, new abnormal elevation of serum creatinine or ≥25% from baseline, or prednisone ≥10 mg daily). Urine specimens were acquired on the day of the biopsy (before the procedure) or within 3 weeks of the kidney biopsy. Serologic features and complement levels were assessed at the clinical visit preceding the biopsy. Proteinuria was measured on or near the day of the biopsy.

### Study Approval

Human study protocols were approved by the institutional review boards (IRBs) at each participating site, and written informed consent was obtained from all participants. For healthy controls, IRB approval was obtained from the Oklahoma Medical Research Foundation. After informed consent, controls were recruited through the Oklahoma Rheumatic Disease Research Cores Center and were matched for sex, race, ethnicity, and age. Subjects were screened using a questionnaire and tested negative for the following antibodies: antinuclear, double-stranded DNA, chromatin, ribosomal P, Ro, La, Smith (Sm), SmRNP, RNP, centromere B, Scl-70, and Jo-1. Samples were processed, stored, and shipped using protocols from the Accelerating Medicines Partnership in Rheumatoid Arthritis and Systemic Lupus Erythematosus (AMP RA/SLE) Network to align with the patient samples.

### Urine Quantibody Assay

An extended version of the Kiloplex Quantibody (RayBiotech) was used to screen urine samples as previously described (35, 63). Concentration of each analyte was normalized by urine creatinine to account for urine dilution. Urine protein abundances are expressed are pgₚᵣₒₜₑᵢₙ/mg_{creatinine}.

### Statistical Analysis

Differential protein abundance in two groups was calculated using a Wilcoxon rank test in univariate analyses. This nonparametric test allowed for robust analysis accounting for the difference in distribution, often not normal, across the 1,200 features. Similar performance to logistic regression (FIG. 12) were observed. For multivariable analyses, linear models or generalized linear models (R lm and glm functions) were used after log transforming the protein abundances (models indicated on top of the figures). To account for sparsity and large variation in the dynamic ranges of the proteins, all values for each protein abundance were added the minimum measured value before log transformation. Correlations and partial correlations (R ppcor package) were calculated on log-transformed protein abundances.

Pathway enrichment analysis was performed with the clusterProfiler or fgsea R packages using the Gene Ontology and Reactome libraries. Genes coding for the measured proteins were used. Analysis was limited to gen sets with at least 5 genes represented in the universe of the 1,200 proteins measured. To account for a limited universe of proteins (not the whole coding genome), self-contained algorithms were applied. GSEA is inherently self-contained. To define the pathways enriched in a distinct group of proteins (i.e., FIG. 22A-D), a hypergeometric test was used. Terms with >75% proteins overlap were removed: the term with the lowest p value was retained. All analyses were performed in R version 4.1.2.

### Results

### Pipeline and Recruitment

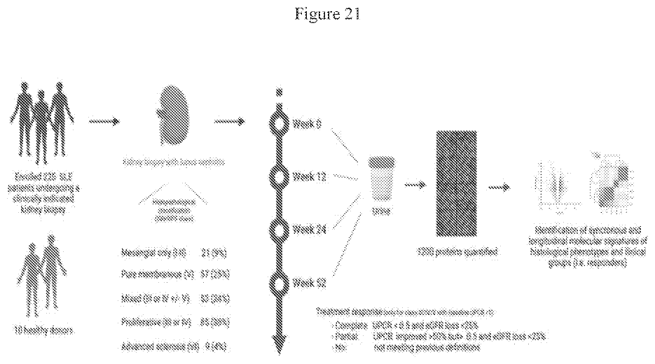

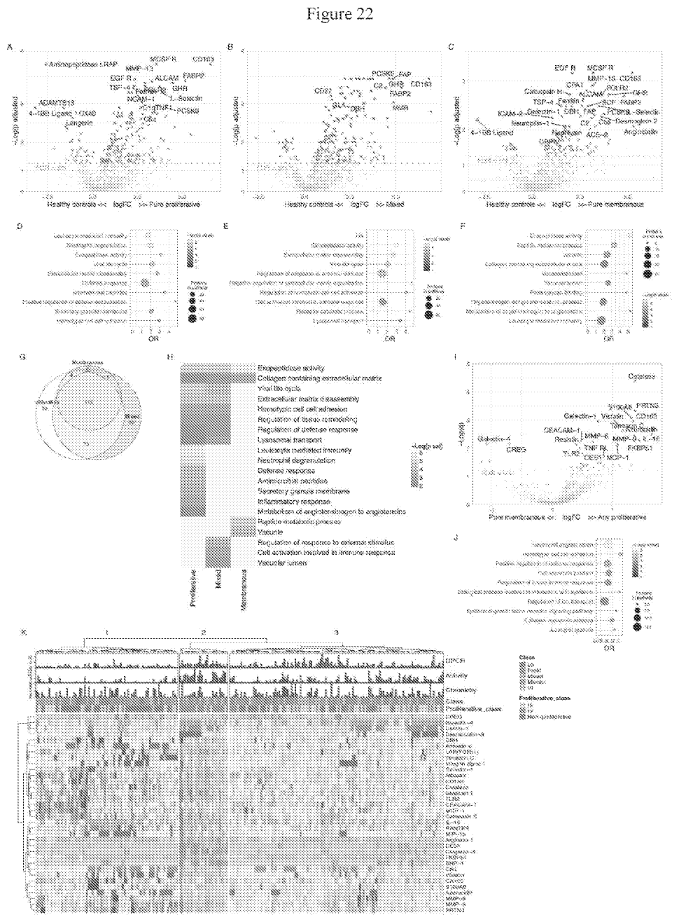

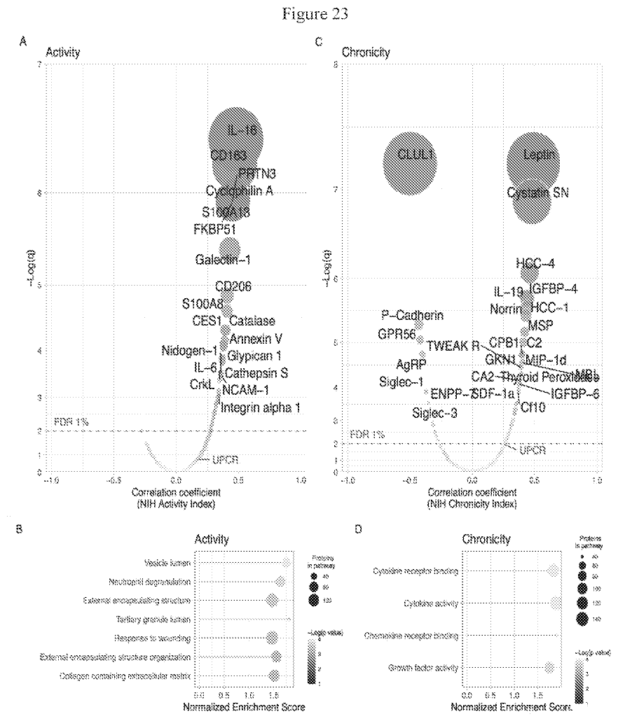

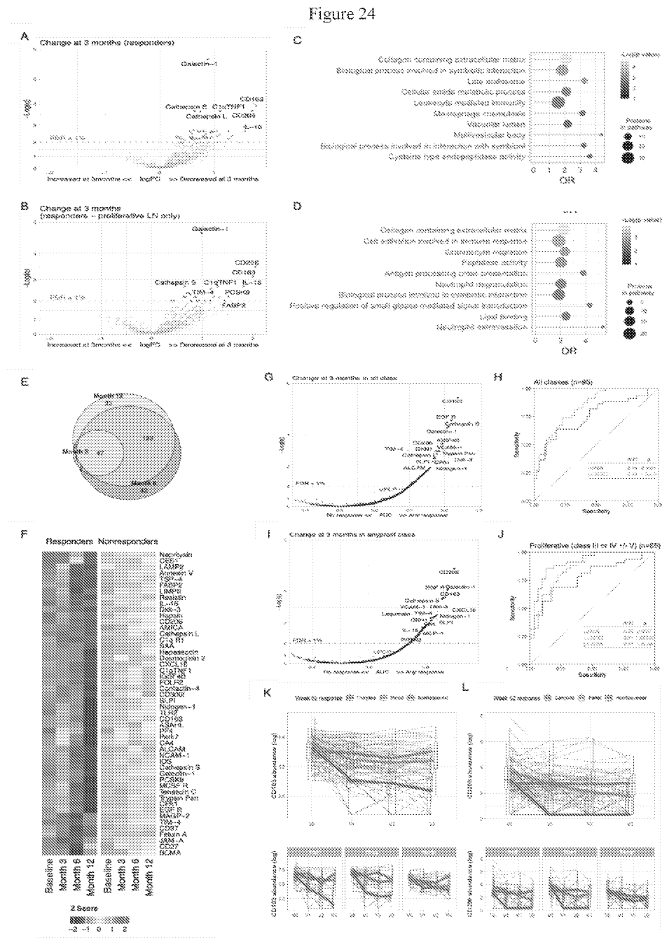

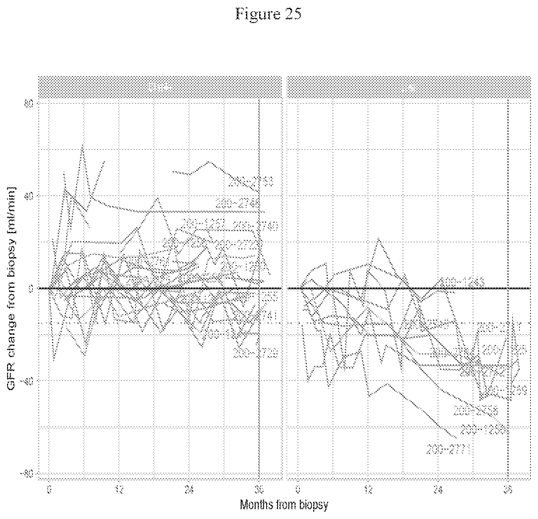

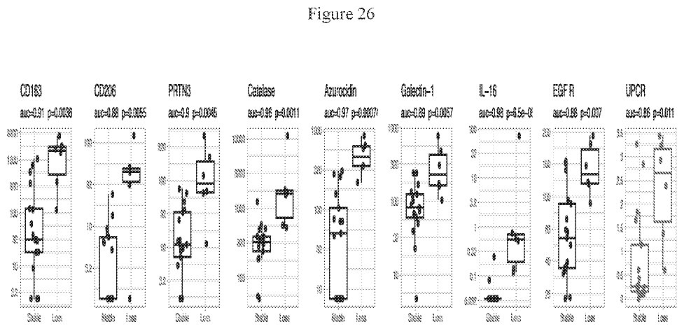

To characterize LN molecular signatures of specific LN subtypes and treatment response, the longitudinal urine proteomic profiles (1200 proteins) of LN patients and their clinical and histologic associations (FIG. 21) are analyzed.

225 SLE patients who underwent a clinically indicated kidney biopsy and had a urine protein to creatinine ratio >0.5 g/g were recruited. To capture LN diversity, all patients with LN were included. Most patients (62%) had proliferative LN: 85 (38%) with pure proliferative LN (class III or IV) and 53 (24%) with mixed LN (class III or IV+V); 25% had pure membranous LN (class V); 21 (9%) had mesangial limited LN (class I or II); and 9 (4%) had advanced sclerosis (class VI). For comparison, 10 healthy donors without past medical history and negative autoimmune serologies were recruited. The baseline clinicodemographic characteristics are summarized by Table D. The patients were similar in age and sex. As expected, proliferative LN had higher histological activity (NIH activity index). Except for class VI (advanced sclerosis), chronicity was similar in the other classes. Proteinuria at the time of biopsy was lower in class I or II LN (median 0.76 [range 0.5-4]) whereas all other classes were similar, highlighting the inability of proteinuria to distinguish between LN classes. The estimated GFR was reduced in all LN patients compared to HD with the lowest values observed in class VI (median 46 ml/min [range 9-63]), followed by proliferative LN (median 88 ml/min [range 12-160]), and pure membranous (median 100 ml/min [range 15-145]). Patients with proliferative or membranous LN were followed longitudinally: complete response rates at week 52 were more common in proliferative LN as compared to pure membranous LN (31% vs 13%). A total of 573 urine proteomic profiles from these 225 unique LN patients and 10 HD were assayed.

TABLE D
Clinical and demographic characteristics

| | Overall | HD | I/II | Proliferative | Mixed | Membranous | VI | p |
|---|---|---|---|---|---|---|---|---|
| n | 235 | 10 | 21 | 85 | 55 | 55 | 9 | |
| Age (mean (SD)) | 36.37 | 38.30 | 39.38 | 34.58 | 36.67 | 36.56 | 41.22 | 0.394 |
| | (11.76) | (14.82) | (11.93) | (11.30) | (11.72) | (11.53) | (13.78) | |
| Sex, F/M (%) | 202/33 | 8/2 | 20/1 | 76/9 | 44/11 | 46/9 | 8/1 | 0.472 |
| | (86.0/14.0) | (80.0/20.0) | (95.2/4.8) | (89.4/10.6) | (80.0/20.0) | (83.6/16.4) | (88.9/11.1) | |
| race (%) | | | | | | | | 0.499 |
| Asian | 35 (14.9) | 1 (10.0) | 5 (23.8) | 13 (15.3) | 8 (14.5) | 6 (10.9) | 2 (22.2) | |
| Black | 99 (42.1) | 3 (30.0) | 11 (52.4) | 32 (37.6) | 18 (32.7) | 30 (54.5) | 5 (55.6) | |
| White | 72 (30.6) | 6 (60.0) | 4 (19.0) | 28 (32.9) | 20 (36.4) | 13 (23.6) | 1 (11.1) | |
| Other | 6 (2.6) | 0 (0.0) | 1 (4.8) | 3 (3.5) | 1 (1.8) | 1 (1.8) | 0 (0.0) | |
| Unknown | 23 (9.8) | 0 (0.0) | 0 (0.0) | 9 (10.6) | 8 (14.5) | 5 (9.1) | 1 (11.1) | |
| First biopsy (%) | 82 (34.9) | 0 (0.0) | 6 (28.6) | 41 (48.2) | 20 (36.4) | 15 (27.3) | 0 (0.0) | 0.002 |
| Proliferative | | | | | | | | <0.001 |
| class (%) | | | | | | | | |
| III | 78 (33.2) | | | 46 (54.1) | 32 (58.2) | | | |
| IV | 60 (25.5) | | | 39 (45.9) | 21 (38.2) | | | |
| NIH Activity | 4.00 [1.00, | | 1.00 [0.00, | 5.00 [3.00, | 7.00 [5.00, | 0.00 [0.00, | 0.00 [0.00, | <0.001 |
| Index (median | 8.00] | | 2.00] | 9.00] | 11.50] | 1.00] | 0.00] | |
| [IQR]) | | | | | | | | |
| NIH Chronicity | 3.00 [1.00, | | 3.00 [2.50, | 2.00 [1.00, | 3.00 [2.00, | 3.00 [1.00, | 9.00 [9.00, | 0.113 |
| Index (median | 5.00] | | 4.00] | 4.00] | 5.25] | 5.75] | 9.00] | |
| [IQR]) | | | | | | | | |
| UPCR (mean | 2.72 (2.39) | | 1.30 (1.09) | 2.39 (1.74) | 3.62 (3.23) | 2.90 (2.42) | 2.16 (1.13) | 0.002 |
| (SD)) | | | | | | | | |
| Serum Cr at | 1.18 (0.82) | 0.76 (0.15) | 1.00 (0.43) | 1.23 (0.91) | 1.21 (0.77) | 1.04 (0.61) | 2.25 (1.37) | 0.001 |
| biopsy (mean | | | | | | | | |
| (SD)) | | | | | | | | |
| eGFR at biopsy | 85.94 | 109.07 | 88.64 | 84.03 | 83.26 | 94.23 | 38.43 | <0.001 |
| (mean (SD)) | (35.93) | (19.11) | (33.58) | (36.34) | (35.25) | (34.74) | (16.69) | |
| Low C3 (%) | 130 (60.2) | | 5 (31.2) | 65 (79.3) | 35 (64.8) | 24 (43.6) | 1 (11.1) | <0.001 |
| Low C4 | 107 (49.5) | | 7 (43.8) | 55 (67.1) | 28 (51.9) | 17 (30.9) | 0 (0.0) | <0.001 |
| Response | | | | | | | | 0.27 |
| status* (%) | | | | | | | | |
| Complete | 33 (25.8) | | | 15 (31.3) | 13 (30.9) | 5 (13.2) | | |
| No | 65 (50.8) | | | 21 (43.8) | 20 (47.6) | 24 (63.2) | | |
| Partial | 30 (23.4) | | | 12 (25) | 9 (21.4) | 9 (23.7) | | |

### Molecular Signatures of Lupus Nephritis

To identify the proteomic signature of each LN class, the urine proteomic profile of LN was compared with healthy controls without clinical proteinuria (FIG. 22A-C). Hundreds of proteins were significantly increased in all LN classes. In this study, pure proliferative LN (class III or IV) and mixed LN (class III or IV+V) are often analyzed together because they share the component of “proliferative” LN which is linked to worse outcomes (67). Accordingly, patients with pure proliferative and mixed LN showed similar proteomic profiles with pathway enrichment analysis detecting leukocyte mediated immunity, viral life cycle, and extracellular matrix disassembly/protease activity (FIG. 22D-H, network analysis in FIG. 7). Most of the proteins enriched in pure membranous LN were also found in proliferative LN indicating common core pathways across proliferative, mixed, and pure membranous LN (FIGS. 22G and 8). In contrast, most of the proteins enriched in proliferative LN (pure or mixed) were not found in membranous suggesting that distinct biological processes are exclusive of proliferative LN. At the pathway enrichment level, all three LN groups showed evidence of protease activity and extracellular matrix remodeling (FIG. 22H).

Because proliferative LN is characterized by an intraglomerular immune infiltrate with endocapillary hypercellularity, the identification of leukocyte mediated immunity proteomic profiles indicates that urine proteomics congruently reflect intrarenal pathology. Proliferative LN is the most aggressive form of LN and carries a higher risk of permanent kidney damage (67). To better define proliferative LN's specific pathological pathways, the proteomic profiles of proliferative (pure and mixed) were compared to pure membranous LN. Proliferative LN signature was dominated by higher levels of CD163 (a macrophage marker), IL-16 (a proinflammatory chemokine), and neutrophil degranulation products such as Catalase, PRTN3, S100A8, Azurocidin, and MMP8 among many others (FIGS. 221 and 7A). Pathway enrichment analysis confirmed that neutrophil degranulation was the biological process most enriched in proliferative LN (FIG. 22J). Several macrophage markers such as CD163, CD206, Galectin-1, and FOLR2 were also enriched in all classes. The urinary abundance of these proteins was similar in pure and mixed proliferative LN but higher than membranous (FIGS. 22F and 8).

The urine abundance of the proteins differentially expressed in the proliferative LN signature is displayed in the heatmap in FIG. 22K. There were 3 clusters of patients defined by low, medium, or high expression of the signature. The “low” (left) cluster included almost exclusively patients with nonproliferative LN. The “medium” (right) cluster included mostly patients with proliferative LN, but also some pure class V, class I/II, and class VI LN. The “high” (middle) cluster identified patients with the highest expression of the proliferative LN signature and was comprised exclusively of patients with proliferative LN. Patients in this cluster were those with the highest activity index in the kidney biopsy and most of those with class IV lupus nephritis.

Altogether, these findings implicate active neutrophil degranulation and macrophage activation in patients with proliferative LN which is stronger in those with higher histological activity. Furthermore, both proliferative and pure membranous LN associated with extracellular matrix degradation. Importantly, these intrarenal biological processes can be noninvasively quantified in the urine.

Histological Activity Correlates with Neutrophil Degranulation and Extracellular Matrix Degradation.

Proliferative LN is heterogeneous in the degree of immunological activity. This is captured by the NIH Activity Index (15). High scores identify more aggressive disease associated with higher risk of kidney failure (67). Five of the six components of the NIH Activity index (endocapillary hypercellularity, neutrophil/karyorrhexis, fibrinoid necrosis, wire loops/hyaline thrombi, and cellular/fibrocellular crescents) are mostly exclusive to proliferative LN (class III or IV+/−V) thereby making the NIH Activity Index a quantitative measure of proliferative LN activity. To characterize the pathways and biomarkers of LN activity, the correlation of the urinary proteins with the NIH Activity Index were studied (FIG. 23A). Several urinary proteins directly correlated with the NIH Activity Index, topped by IL-16 and CD163 (FIG. 23A) were found. As supported by pathway enrichment analysis (FIG. 23B), the signature of LN activity also included proteins associated with neutrophil degranulation (i.e., PRTN3, Azurocidin, Visfatin, MMP8, LAMP1, Catalase), macrophage activation (i.e., CD163, CD206, Galectin-1), and wound healing/matrix degradation (i.e., Nidogen-1, Decorin) (FIG. 9). Importantly, these associations persisted after adjusting for proteinuria or renal fibrosis (NIH Chronicity Index) in a multivariable model (FIG. 10). These findings further support the link between proliferative LN and both myeloid activity and wound healing pathways by demonstrating a direct quantitative association with proliferative LN activity, independently of proteinuria.

Next, the proteomic correlates of intrarenal damage as quantified by the NIH Chronicity Index were studied. The NIH Chronicity Index captures features of irreversible damage such as interstitial fibrosis and tubular damage, glomerulosclerosis, and fibrous crescents. FIG. 23C displays the urinary proteins positively and negatively correlated with intrarenal chronicity. Pathway enrichment analysis identified cytokine/chemokines and grow factor activity (FIG. 23D). These associations persisted after adjusting for proteinuria and the NIH Activity Index (FIG. 10).

Treatment Response is Associated with a Decline of Urinary Biomarkers of LN Activity Including Markers of Myeloid Immunity and Matrix Degradation.

Next, the proteomic signatures linked to treatment response were focused. Complete renal response is currently defined by a decline of urine protein-to-creatinine ratio (UPCR) to <0.5 after 1 or 2 years. It is associated with better long-term preservation of kidney function in LN. To assess response, a baseline UPCR >1 was required (68). In this analysis, a total of 127 patients were included: 48 (38%) with pure proliferative LN (class III or IV), 41 (32%) with mixed LN (III or IV+/−V), and 38% (30%) with pure membranous LN. Response was complete in 34 (27%), partial in 29 (23%), and none in 64 (50%). In this cohort, treatment was at the discretion of the treating physician.

At the time of kidney biopsy (baseline), there was no difference in the urinary proteomic profiles in patients who achieved clinical response at 1 year (responders) compared to nonresponders (FIG. 11), even when the analysis was restricted to patients treated with the same regimen, mycophenolate. Therefore, longitudinal trajectories were focused.

To identify the pathways that could mediate response to immunosuppression, the changes in the urinary proteome after 3 months of treatment compared to the baseline were studied, according to the response status. Patients who responded at 1 year showed a decline at 3 months in 51 urinary proteins (FDR <1%) led by Galectin-1, CD163, IL-16, and macrophage mannose receptor (CD206) (FIG. 24A-B). These proteins overlapped with the proteomic signature associated with histological activity (FIG. 23A). Accordingly, pathway enrichment analysis at 3 months into treatment showed a decline in pathways related to extracellular matrix and cellular immune response in those who ultimately had a complete or partial response at 1 year (FIG. 24C-D).

The decline of the urine proteins reduced at 3 months persisted at 6 and 12 months, while even more proteins declined at 6 and 12 months (FIGS. 24E-F and 11B-E). By contrast, there were no changes observed in nonresponders (FIG. 24F).

To identify early biomarkers of response, the discriminatory ability of the urinary protein changes at 3 months to predict 1 year response was studied. One-hundred urinary biomarkers predicted response (FDR <1%, AUC 0.7-0.84), all outperforming the improvement of UPCR (clinical standard) (FIG. 24G). A decline of CD163 predicted 1 year response in ROC analysis with an area under the curve (AUC) of 0.84 (q=1.7*10-5) compared to an AUC of 0.69 (q=0.015) for UPCR decline (FIG. 24H). In proliferative LN, urinary biomarkers displayed superior performance with an AUC of 0.91 (q=2*10-5), 0.86 (q=1.7*10⁻⁴), and 0.78 (q=0.01) for the decline of CD206, CD163, and UPCR, respectively (FIG. 24I-J). There were no biomarker changes at 3 months predicting response in pure membranous LN that reached statistical significance after correcting for multiple comparisons. Testican-1, IL-31 RA, and CD163 displayed AUCs ranging 0.74-0.8 with nominal p values <0.05 (q=0.89 for all).

These findings indicate that effective immunosuppression induces by 3 months an immunological response in the kidney that can be noninvasively monitored in the urine. Because proliferative LN is characterized by the infiltration of intraglomerular myeloid immune cells, a decline in urinary biomarkers of myeloid inflammation in responders suggests a parallel resolution of intraglomerular inflammation. This was specific to proliferative LN as exemplified by the trajectories of CD163 and CD206 (FIG. 24K-L).

### Conclusions

Precision medicine relies on the detection of mechanistically anchored disease states tied to clinically relevant outcomes. The discovery of disease mechanisms, patient subgroups, biomarkers, and novel targets can be simultaneously derived from the analysis of careful phenotypes, longitudinal trajectories, and differential outcomes associated with specific interventions (69). In the present disclosure, it is defined that neutrophil degranulation, macrophage activation, and extracellular matrix degradation are implicated in LN activity. These processes can be noninvasively quantified and monitored in urine. Improvement of these pathways occurred at 3 months and predicted treatment response. These noninvasive urine biomarkers (such as CD163 and CD206) that parallel intrarenal inflammation outperform the current clinical standard (proteinuria). Furthermore, this study validated IL-16 as the urinary biomarker most correlated with LN activity (63) supporting its role as a novel therapeutic target and biomarker.

The current classification and treatment of LN rely on histological features at the time of biopsy. A higher NIH Activity Index usually triggers more aggressive immunosuppression. In contrast, when there is a low NIH Activity Index in the presence of a high NIH Chronicity Index, proteinuria is considered secondary to damage and, therefore, not requiring new or increased immunosuppression. Importantly, persistent elevation of the NIH Activity Index in a repeat biopsy is associated with LN flares and 44% 10-year kidney survival, compared to 100% in patients with an index of 0, regardless of resolution of proteinuria (3, 27, 36, 70). Therefore, the characterization of the pathways involved in LN activity is key to the identification of new treatable targets and biomarkers to guide diagnosis and treatment. Currently, there has been no way to do frequent kidney biopsies to judge changes in activity and chronicity indices. The present application provides that proteomic analysis offered a detailed view of the pathways and biomarkers linked to LN activity frequently over time, with changes at 3 months predictive of treatment response at later time points. Patients with higher activity had higher urinary abundance of biomarkers of inflammation (i.e., IL-16), neutrophil degranulation (i.e., PRTN3, Azurocidin, Catalase, MMP8, LAMP1-2), macrophage activation (i.e., CD163, CD206, Galectin-1, Cathepsins, MIP-1b), and extracellular matrix degradation (i.e., Nidogen-1, collagens, proteoglycans). Longitudinally, a reduction in biomarkers of these processes predicted future treatment response. This suggests that the effective inhibition of pathogenic mechanisms by immunosuppression can be noninvasively monitored in real time. These responses are faster than the resolution of proteinuria which requires slower kidney repair. A biomarker panel to noninvasively assess intrarenal activity may reshape the treatment strategy of LN based on “immunological responses”. For example, patients with persistent urinary biomarker elevation (indicating activity regardless of improved proteinuria) would receive stronger, different, or prolonged immunosuppression, while those with normal urinary biomarker levels (indicating immunologically resolved LN) could continue and eventually safely taper these potentially toxic medications.

Histological activity correlated with a macrophage signature. Macrophages are the dominant immune cell type in LN. Intraglomerular and tubulointerstitial macrophages are abundant in proliferative LN as compared to pure class V and II (54). The intrarenal macrophage subsets in LN are heterogeneous (71), but most are “alternatively activated” (M2) as opposed to inflammatory (M1) (54). M2 macrophages functions include repair/pro-fibrotic (M2a), immune regulation (M2b), and anti-inflammatory/scavenging/apoptosis-clearance/pro-fibrotic (M2c) (53, 72, 73). M2c, followed by M2a, macrophages are the most abundant type in LN (54) and are associated with injury. Important to this findings, M2c macrophages express CD163 and CD206 (72). In this analysis, urinary CD163 and CD206 were increased in all classes (but at higher levels in proliferative LN), they correlated with the NIH Activity Index, and their decline best predicted treatment response. Similarly, the intrarenal abundance of CD163+ and CD206+ macrophages correlated with LN histopathological indices of LN activity (54, 55). Intrarenal CD163+ macrophages with phagocytic, apoptosis-clearing, and repair phenotypes were also identified in LN by single cell RNA sequencing by the group (57) (and are associated with LN activity). Urinary galectin-1 was also linked to histological activity and, its decline, with treatment response. Galectin-1 has several functions including promoting an anti-inflammatory/proresolving M2 macrophage phenotype (74). In neutrophils, galectin-1 inhibits activation, chemotaxis, and extravasation while favoring phagocytic removal of viable neutrophils (74). These findings (i) confirm the association between injury-associated macrophages and LN activity and (ii) indicate that the disappearance of these cells or their differentiation to a different phenotype anticipates better outcomes. Whether these cells are primary drivers of LN or responders to another process causing kidney injury remains to be studied.

In this study, it was observed that neutrophil granule content (i.e., PR3 and Azurocidin) in the urine was linked to LN activity implicating neutrophil degranulation in proliferative LN. Neutrophils, especially the subset of low-density granulocytes, have been widely implicated in SLE pathogenesis and LN. Blood transcriptome studies revealed that LN is associated with higher expression of neutrophil-associated transcriptional profiles (31, 75, 76). Intraglomerular neutrophils and karyorrhectic debris from apoptotic neutrophils are in fact a feature of proliferative LN and are scored in the NIH Activity Index (15). However, mature neutrophils with classical polylobate nuclei are not a dominant cell type observed in LN kidney biopsies. Immature forms of neutrophils identified among low density granulocytes have been implicated in the pathogenesis of LN (77, 78). These cells did not have polylobate nuclei suggesting that their presence in LN kidney may not be noted with traditional light microscopy (78). These less mature forms of granulocytes have enhanced ability to degranulate (78) suggesting an active role in LN. PR3 can in fact lead to extracellular matrix degradation which, in turn, can lead to fibrosis and irreversible kidney damage (79-81). Furthermore, neutrophil extracellular traps (NETs) were demonstrated in the glomeruli of patients with proliferative LN and the percentage of glomeruli infiltrated by netting neutrophils correlated with the NIH Activity index (32). Collectively, these results indicate that neutrophil degranulation is occurring in active LN and can be noninvasively monitored in the urine. Moreover, degranulating neutrophils and phagocytic/pro-repair macrophages cooccur in active LN further suggesting ongoing interaction. In the initial stages of LN, immune complexes and neutrophils help to recruit inflammatory and patrolling macrophages (10). But as these start to phagocytize damaged material they become less inflammatory and more phagocytic/reparative and move into crescents and surrounding tissue (71). This model suggests that neutrophil activation is an early driver of kidney inflammation in LN. Novel precision medicine studies showed that the identification SLE patients in neutrophil-driven subsets may guide treatment selection (82). Future longitudinal studies should determine how specific treatments such as steroids can be titrated according to an active degranulation signature. The optimization of the balance between pro-inflammatory and pro-resolving myeloid cells could lead to resolution of kidney-damaging inflammation while curtailing pro-fibrotic stimuli.

As described in the present application, urinary IL-16 is the protein most correlated with the NIH Activity Index (63). This finding is validated by applying an unbiased approach in an independent larger cohort of LN patients, corroborating the role of IL-16 both as a clinical biomarker and as a participant in LN pathogenesis. IL16 polymorphisms have been associated with increased risk of SLE (OR 3.3-10.4) suggesting a potential causal role (47). IL-16 is a proinflammatory chemokine secreted by immune cells and nonimmune cells in response to several stimuli, such as complement activation, antigen stimulation, interferon, hypoxia, cell injury, and apoptosis (39, 42, 43). Pro-IL-16 is cleaved into bioactive IL-16 by caspase 3 (43) or PR3 (45) indicating that both cell death and neutrophil degranulation may lead to IL-16 activation. IL-16 is a ligand for CD4 (83) and CD9 (84). In addition to T cells, neutrophil progenitors express CD4 (85) and so do circulating neutrophils in some individuals (86) suggesting that IL-16 may attract and activate several cell types, including immature neutrophils to the kidney. Furthermore, CD9 controls migration and proliferation of parietal epithelial cells in response to podocyte injury (87). CD9 stimulation mediates glomerular crescent formation and glomerular demolition (87), thereby linking IL-16 to a non-immune mechanism of proliferative LN. Crescents are associated with poor renal survival and mortality in LN (88, 89).

Collectively, the findings from this work indicate that following an inciting event such as immune complex deposition (10), the active phase of proliferative LN is characterized by neutrophil degranulation, phagocytic/injury-associated macrophage activation, chemokine release, and extracellular matrix degradation. IL-16 may be playing a central role fueling inflammation by attracting more immune cells such as neutrophils and promoting crescent formation. Neutrophil degranulation may directly damage the glomerular endothelium (90) and remodel extracellular matrix promoting chronic kidney disease. It is unclear whether phagocytic and injury-associated macrophages play a regulatory or proinflammatory role in the initial phase of LN activity. Nevertheless, their disappearance or their differentiation to a different phenotype is associated with treatment response suggesting that they track with the resolution of inflammation. Importantly, these pathogenic processes can be noninvasively monitored in the urine.

Prediction of treatment response is key to improve treatment strategies. Although there were no biomarkers at baseline that predicted response, the decline of several urinary biomarkers after 3 months of treatment strongly predicted response at 1 year. These findings underscore the power of individual trajectories to discover disease biology and to identify clinically meaningful patient subsets. On one hand, therapeutic interventions can serve as molecular scalpels to discover the biological changes that underlie treatment response. On the other hand, this study revealed powerful biomarkers of early response. These biomarkers may guide treatment selection and clinical trial design. For example, there are currently several treatment options for LN, but the choice of the best initial treatment strategy remains unclear. In patients where urine proteomics showed no reduction in these predictors of treatment response, treatment could be rapidly modified until an immunological response is achieved without waiting for improvement in proteinuria. It is shown that proteinuria does not track with intrarenal inflammation. Conversely, early immunological responses in the urine proteome can reassure that the current treatment is effective. In the present disclosure, this study demonstrates the ability of urine proteomics to explore a wide array of pathological processes, including neutrophil biology which can be missed in cellular studies involving sample freezing (57). Future longitudinal studies should address how these urinary biomarkers of intrarenal pathology can guide treatment and whether immunological responses predict long term preservation of kidney function.

In the present disclosure, this study confirmed several known biomarkers of LN. Among others, urinary CD163(37, 63, 91), MCP-1(92), Lipocalin-2 (93), and ALCAM (11) were increased in proliferative LN. Of these, only CD163 and MCP-1 correlated with the NIH Activity Index. EGF-R (94) negatively correlated with the NIH Chronicity Index and positively with the NIH Activity Index.

### Example 5: Urinary Biomarkers at 1 Year Predict Kidney Function Loss at 3 Years

Lupus nephritis (LN) is a severe complication of systemic lupus erythematosus associated with end-stage kidney disease and mortality. Diagnosis is based on a kidney biopsy in patients with abnormal urine protein amount (“proteinuria”). Treatment involves immunosuppression. Response to treatment is determined by a reduction of proteinuria below 0.5 g/24 h (or 0.5 gₚᵣₒₜₑᵢₙ/g_{creatinine}) usually measured after 1 year of treatment. However, proteinuria is an inadequate biomarker. About 50% of patients with proteinuria <0.5 have persistently active LN on kidney biopsy and 62% of patients with inactive LN on biopsy have proteinuria >0.5 (Malvar et al., Nephrol Dial Transplant 2017). Proteinuria at 1 year is used because partially predicts long term outcomes. Proteinuria <0.7 at 1 year is associated with lower risk of irreversible loss of kidney function at 7 years (Dall'Era et al., Arthritis Rheumatol 2015).

Noninvasive urinary biomarkers of LN activity were discovered. It was also shown that a reduction of these biomarkers after 3 months of treatment predict proteinuric response at 12 months with an AUC up to 0.91 for a single biomarker.

Urinary biomarkers of lupus nephritis (LN) have been studied for >20 years yet are not part of clinical practice. It was reasoned that if it was demonstrated that urinary biomarkers predict irreversible loss of kidney function (a “hard outcome”) outperforming the currently available standard biomarkers, there would be a strong drive to bring this tool into clinical practice and clinical trial design. Especially because their use could guide treatment and prevent kidney function loss.

In preliminary analysis, 31 LN patients at Johns Hopkins for at least 3 years after their diagnostic kidney biopsy were followed. 1200 urinary biomarkers were quantified at time of biopsy and after 3, 6, and 12 months. Patients were classified as having kidney function loss if their glomerular filtration rate (GFR) declined by >15 ml/min after 3 years (FIG. 25). This is a steep GFR slope defining a substantial amount of kidney function loss. The GFR slope is now the recommended biomarker of choice by the FDA and EMA (Levey et al., AJKD 2020). About 30% of patients showed GFR loss at 3 years.

It is hypothesized that persistent elevation of candidate urinary biomarkers of LN activity after 1 year of treatment would indicate inadequate response to immunosuppression. Therefore, because untreated active LN leads to GFR loss, patients with persistently elevated biomarkers of active LN would develop GFR loss. FIG. 26 displays that the persistent elevation of biomarkers of LN activity predicts at 1 year predict GFR loss at 3 years, better than proteinuria, the clinical standard. These biomarkers can be combined along with other clinical variables into a panel to further improve their performance.

In patients with elevated proteinuria at 1 year, a repeat kidney biopsy would be normally considered to determine if the patient has persistent inflammation requiring a different immunosuppressive treatment or if they developed kidney scarring causing proteinuria but not requiring immunosuppression. With the novel biomarkers, persistent elevation of biomarkers of LN activity (=inflammation) may indicate the need for more immunosuppression and prevent a kidney biopsy. These new results demonstrate that 1) these biomarkers at 1 year predict kidney function at 3 years and 2) they indicate kidney inflammation. Therefore, these biomarkers could be used as surrogate endpoints in clinical trials and as clinical biomarkers in clinical practice. This would shift the field from the imperfect proteinuric response to a more biologically grounded and accurate immunological response.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### REFERENCES

All references, including publications, patent applications, and patents, cited herein are hereby incorporated by reference to the same extent as if each reference were individually and specifically indicated to be incorporated by reference and were set forth in its entirety herein.
1. Fava, A. & Petri, M. Systemic lupus erythematosus: Diagnosis and clinical management. J. Autoimmun. 96, 0-1 (2018).
2. Costenbader, K. H. et al. Trends in the incidence, demographics, and outcomes of end-stage renal disease due to lupus nephritis in the US from 1995 to 2006. Arthritis Rheum. 63, 1681-1688 (2011).
3. De Rosa, M. et al. A prospective observational cohort study highlights kidney biopsy findings of lupus nephritis patients in remission who flare following withdrawal of maintenance therapy. Kidney Int. 94, 788-794 (2018).
4. Greloni, G. et al. Value of repeat biopsy in lupus nephritis flares. Lupus Sci. Med. 1, 1-6 (2014).
5. Luciano, R. L. & Moeckel, G. W. Update on the Native Kidney Biopsy: Core Curriculum 2019. Am. J. Kidney Dis. 73, 404-415 (2019).
6. Geldenhuys, L. et al. Percutaneous native renal biopsy adequacy: A successful interdepartmental quality improvement activity. Can. J. Kidney Heal. Dis. 2, 1-7 (2015).
7. Birmingham, D. J. et al. Biomarkers of lupus nephritis histology and flare: deciphering the relevant amidst the noise. Nephrol. Dial. Transplant 32, i71-i79 (2017).
8. Soliman, S. & Mohan, C. Lupus nephritis biomarkers. Clin. Immunol. 185, 10-20 (2017).
9. Pejchinovski, M. et al. Urine peptidomic biomarkers for diagnosis of patients with systematic lupus erythematosus. Lupus 27, 6-16 (2018).
10. Davidson, A. What is damaging the kidney in lupus nephritis? Nat. Rev. Rheumatol. 12, 143-153 (2016).
11. Stanley, S. et al. Comprehensive aptamer-based screening identifies a spectrum of urinary biomarkers of lupus nephritis across ethnicities. Nat. Commun. 11, 2197 (2020).
12. Zhang, T., Duran, V., Vanarsa, K. & Mohan, C. Targeted urine proteomics in lupus nephritis—a meta-analysis. Expert Rev. Proteomics 17, 767-776 (2020).
13. Vanarsa, K. et al. Quantitative planar array screen of 1000 proteins uncovers novel urinary protein biomarkers of lupus nephritis. Ann. Rheum. Dis. 79, 1349-1361 (2020).
14. Platonov, M. E. et al. KISS1 tumor suppressor restricts angiogenesis of breast cancer brain metastases and sensitizes them to oncolytic virotherapy in vitro. Cancer Lett. 417, 75-88 (2018).
15. Bajema, I. M. et al. Revision of the International Society of Nephrology/Renal Pathology Society classification for lupus nephritis: clarification of definitions, and modified National Institutes of Health activity and chronicity indices. Kidney Int. 93, 789-796 (2018).
16. Arazi, A. et al. The immune cell landscape in kidneys of patients with lupus nephritis. Nat. Immunol. 20, 902-914 (2019).
17. Nguyen, T. M., Shafi, A., Nguyen, T. & Draghici, S. Identifying significantly impacted pathways: A comprehensive review and assessment. Genome Biol. 20, 1-15 (2019).
18. Subramanian, A. et al. Gene set enrichment analysis: A knowledge-based approach for interpreting genome-wide expression profiles. Proc. Natl. Acad. Sci. U.S.A 102, 15545-15550 (2005).
19. Kuleshov, M. V et al. Enrichr: a comprehensive gene set enrichment analysis web server 2016 update. Nucleic Acids Res. 44, W90-7 (2016).
20. Ritchie, M. E. et al. limma powers differential expression analyses for RNA-sequencing and microarray studies. Nucleic Acids Res. 43, e47-e47 (2015).
21. Turck, N. et al. pROC: an open-source package for R and S+ to analyze and compare ROC curves. BMC Bioinformatics 8, 12-77 (2011).
22. R Core Team. R: A Language and Environment for Statistical Computing. (2018).
23. Wang, Y. et al. iTALK: an R Package to Characterize and Illustrate Intercellular Communication. bioRxiv 507871 (2019). doi:10.1101/507871
24. IUPHAR/BPS. No Title. Guide to Pharmacology (2019).
25. Sloan, R. P., Schwartz, M. M., Korbet, S. M. & Borok, R. Z. Long-term outcome in systemic lupus erythematosus membranous glomerulonephritis. Lupus Nephritis Collaborative Study Group. J. Am. Soc. Nephrol. 7, 299-305 (1996).
26. Norden, A. G. W., Lapsley, M. & Unwin, R. J. Urine retinol-binding protein 4. A functional biomarker of the proximal renal tubule. Advances in Clinical Chemistry 63, (Elsevier Inc., 2014).
27. Alvarado, A. S. et al. The value of repeat kidney biopsy in quiescent Argentinian lupus nephritis patients. Lupus 23, 840-847 (2014).
28. Wu, H. et al. Single-cell transcriptomics of a human kidney allograft biopsy specimen defines a diverse inflammatory response. J. Am. Soc. Nephrol. 29, 2069-2080 (2018).
29. Wu, H. et al. Comparative Analysis and Refinement of Human PSC-Derived Kidney Organoid Differentiation with Single-Cell Transcriptomics. Cell Stem Cell 23, 869-881.e8 (2018).
30. Muto, Y., Wilson, P. C., Wu, H., Waikar, S. S. & Humphreys, B. Single cell transcriptional and chromatin accessibility profiling redefine cellular heterogeneity in the adult human kidney. bioRxiv 2020.06.14.151167 (2020). doi:10.1101/2020.06.14.151167
31. Banchereau, R. et al. Personalized Immunomonitoring Uncovers Molecular Networks that Stratify Lupus Patients. Cell 165, 551-565 (2016).
32. Villanueva, E. et al. Netting Neutrophils Induce Endothelial Damage, Infiltrate Tissues, and Expose Immunostimulatory Molecules in Systemic Lupus Erythematosus. J. Immunol. 187, 538-552 (2011).
33. Linge, P., Fortin, P. R., Lood, C., Bengtsson, A. A. & Boilard, E. The non-haemostatic role of platelets in systemic lupus erythematosus. Nat. Rev. Rheumatol. 14, 195-213 (2018).
34. Wei, R. et al. Alterations in urinary collagen peptides in lupus nephritis subjects correlate with renal dysfunction and renal histopathology. Nephrol. Dial. Transplant. 32, 1468-1477 (2017).
35. Fava, A. et al. Integration of urine proteomics and renal single-cell genomics identifies an interferon-gamma response gradient in lupus nephritis. JCI Insight in press, (2020).
36. Malvar, A. et al. Histologic versus clinical remission in proliferative lupus nephritis. Nephrol. Dial. Transplant. 32, 1338-1344 (2017).
37. Mejia-Vilet, J. M. et al. Urinary Soluble CD163: a Novel Noninvasive Biomarker of Activity for Lupus Nephritis. J. Am. Soc. Nephrol. ASN.2019121285 (2020). doi:10.1681/ASN.2019121285
38. Torabinejad, S. et al. Urinary monocyte chemotactic protein-1 and transforming growth factor-O in systemic lupus erythematosus. Indian J. Nephrol. 22, 5-12 (2012).
39. Glass, W. G., Sarisky, R. T. & Del Vecchio, A. M. Not-so-sweet sixteen: The role of IL-16 in infectious and immune-mediated inflammatory diseases. J. Interf Cytokine Res. 26, 511-520 (2006).
40. Wu, D. M. H. et al. Processing and release of IL-16 from CD4+ but not CD8+ T cells is activation dependent. J. Immunol. 162, 1287-1293 (1999).
41. Bellini, A., Yoshimura, H., Vittori, E., Marini, M. & Mattoli, S. Bronchial epithelial cells of patients with asthma release chemoattractant factors for T lymphocytes. J. Allergy Clin. Immunol. 92, 412-424 (1993).
42. Roth, S. et al. Secondary necrotic neutrophils release interleukin-16C and macrophage migration inhibitory factor from stores in the cytosol. Cell Death Discov. 1, 1-9 (2015).
43. Zhang, Y. et al. Processing and activation of pro-interleukin-16 by caspase-3. J. Biol. Chem. 273, 1144-1149 (1998).
44. Cruikshank, W. & Little, F. Interleukin-16: The ins and outs of regulating T-cell activation. Crit. Rev. Immunol. 28, 467-483 (2008).
45. Kerstein-Staehle, A. et al. OP0054 New role for proteinase 3 in IL-16 bioactivity control in granulomatosis with polyangiitis. Ann. Rheum. Dis. 80, 28 LP-29 (2021).
46. Lun, W. H. et al. Loss of virus-specific CD4+ T cells with increases in viral loads in the chronic phase after vaccine-based partial control of primary simian immunodeficiency virus replication in macaques. J. Gen. Virol. 85, 1955-1963 (2004).
47. Xue, H. et al. The IL-16 gene polymorphisms and the risk of the systemic lupus erythematosus. Clin. Chim. Acta 403, 223-225 (2009).
48. Lard, L. R., Roep, B. O., Verburgh, C. A., Zwinderman, A. H. & Huizinga, T. W. J. Elevated IL-16 levels in patients with systemic lupus erythematosus are associated with disease severity but not with genetic susceptibility to lupus. Lupus 11, 181-185 (2002).
49. Mariani, L. H. et al. Interstitial fibrosis scored on whole-slide digital imaging of kidney biopsies is a predictor of outcome in proteinuric glomerulopathies. Nephrol. Dial. Transplant. 33, 310-318 (2018).
50. de Oliveira, J. G. G., Xavier, P. D. P., Sampaio, S. M., Tavares, I. S. & Mendes, A. A. The synthesis by fine-needle aspiration biopsy cultures of IL-7, IL-16 and IL-18 is significantly associated with acute rejection in kidney transplants. Nephron 92, 622-628 (2002).
51. Kawabata, K. et al. IL-16 expression is increased in the skin and sera of patients with systemic sclerosis. Rheumatol. (United Kingdom) 59, 519-523 (2020).
52. Fava, A., Raychaudhuri, S. & Rao, D. A. The Power of Systems Biology. Rheum. Dis. Clin. North Am. 1-16 (2021). doi:10.1016/j.rdc.2021.04.003
53. Lu, J. et al. Discrete functions of M2a and M2c macrophage subsets determine their relative efficacy in treating chronic kidney disease. Kidney Int. 84, 745-755 (2013).
54. Olmes, G. et al. CD163+M2c-like macrophages predominate in renal biopsies from patients with lupus nephritis. Arthritis Res. Ther. 18, 1-16 (2016).
55. Li, J., Yu, Y. F., Liu, C. H. & Wang, C. M. Significance of M2 macrophages in glomerulonephritis with crescents. Pathol. Res. Pract. 213, 1215-1220 (2017).
56. Li, J., Liu, C. H., Xu, D. L. & Gao, B. Significance of CD163-positive macrophages in proliferative glomerulonephritis. Am. J. Med. Sci. 350, 387-392 (2015).
57. Arazi, A. et al. The immune cell landscape in kidneys of patients with lupus nephritis. Nat. Immunol. 20, 902-914 (2019).
58. Avihingsanon, Y. et al. Measurement of urinary chemokine and growth factor messenger RNAs: A noninvasive monitoring in lupus nephritis. Kidney Int. 69, 747-753 (2006).
59. Tsakas, S. & Goumenos, D. S. Accurate measurement and clinical significance of urinary transforming growth factor-beta. Am. J. Nephrol. 26, 186-193 (2006).
60. Warstat, K. et al. Transforming growth factor β1 and laminin-111 cooperate in the induction of interleukin-16 expression in synovial fibroblasts from patients with rheumatoid arthritis. Ann. Rheum. Dis. 69, 270-275 (2010).
61. Lee, J. S., Lim, J. Y. & Kim, J. Mechanical stretch induces angiotensinogen expression through PARP1 activation in kidney proximal tubular cells. Vitr. Cell. Dev. Biol.—Anim. 51, 72-78 (2015).
62. Etzerodt et al., Antioxid. Redox Signal 18(17):2352-2363 (2013)
63. Fava A, Rao D A, Mohan C, Zhang T, Rosenberg A, Fenaroli P, Belmont H M, Izmirly P, Clancy R, Trujillo J M, Fine D, Arazi A, Berthier C C, Davidson A, James J A, Diamond B, Hacohen N, Wofsy D, Raychaudhuri S, Apruzzese W, Buyon J, Petri M. Urine Proteomics and Renal Single-Cell Transcriptomics Implicate Interleukin-16 in Lupus Nephritis. Arthritis Rheumatol. 2022; 74(5):829-39. Epub 20220416. doi: 10.1002/art.42023. PubMed PMID: 34783463; PMCID: PMC9050800.
64. Nicolaou O, Kousios A, Hadjisavvas A, Lauwerys B, Sokratous K, Kyriacou K. Biomarkers of systemic lupus erythematosus identified using mass spectrometry-based proteomics: a systematic review. J Cell Mol Med. 2017; 21(5):993-1012. Epub 20161123. doi: 10.1111/jcmm.13031. PubMed PMID: 27878954; PMCID: PMC5387176.
65. Zhang X, Jin M, Wu H, Nadasdy T, Nadasdy G, Harris N, Green-Church K, Nagaraja H, Birmingham D J, Yu C Y, Hebert L A, Rovin B H. Biomarkers of lupus nephritis determined by serial urine proteomics. Kidney Int. 2008; 74(6):799-807. Epub 20080702. doi: 10.1038/ki.2008.316. PubMed PMID: 18596723; PMCID: PMC2614389.
66. Fava A, Raychaudhuri S, Rao D A. The Power of Systems Biology: Insights on Lupus Nephritis from the Accelerating Medicines Partnership. Rheum Dis Clin North Am. 2021; 47(3):335-50. Epub 20210616. doi: 10.1016/j.rdc.2021.04.003. PubMed PMID: 34215367.
67. Austin H A, 3rd, Muenz L R, Joyce K M, Antonovych T T, Balow J E. Diffuse proliferative lupus nephritis: identification of specific pathologic features affecting renal outcome. Kidney Int. 1984; 25(4):689-95. doi: 10.1038/ki.1984.75. PubMed PMID: 6482173.
68. Carlucci P M, Li J, Fava A, Deonaraine K K, Wofsy D, James J A, Putterman C, Diamond B, Davidson A, Fine D M, Monroy-Trujillo J, Atta M G, DeJager W, Guthridge J M, Haag K, Rao D A, Brenner M B, Lederer J A, Apruzzese W, Belmont H M, Izmirly P M, Zaminski D, Wu M, Connery S, Payan-Schober F, Furie R, Dall'Era M, Cho K, Kamen D, Kalunian K, Anolik J, Barnas J, Ishimori M, Weisman M H, Accelerating Medicines Partnership RASLEN, Buyon J P, Petri M. High incidence of proliferative and membranous nephritis in SLE patients with low proteinuria in the Accelerating Medicines Partnership. Rheumatology (Oxford). 2022. Epub 20220225. doi: 10.1093/rheumatology/keac067. PubMed PMID: 35212719.
69. Rosen A, Zeger S L. Precision medicine: discovering clinically relevant and mechanistically anchored disease subgroups at scale. J Clin Invest. 2019; 129(3):944-5. Epub 20190128. doi: 10.1172/JCI126120. PubMed PMID: 30688662; PMCID: PMC6391083.
70. Alsuwaida A, Husain S, Alghonaim M, AlOudah N, Alwakeel J, ullah A, Kfoury H. Strategy for second kidney biopsy in patients with lupus nephritis. Nephrol Dial Transplant. 2012; 27(4):1472-8. Epub 20110919. doi: 10.1093/ndt/gfr517. PubMed PMID: 21931127.
71. Davidson A. Renal Mononuclear Phagocytes in Lupus Nephritis. ACR Open Rheumatol. 2021; 3(7):442-50. Epub 20210601. doi: 10.1002/acr2.11269. PubMed PMID: 34060247; PMCID: PMC8280821.
72. Anders H J, Ryu M. Renal microenvironments and macrophage phenotypes determine progression or resolution of renal inflammation and fibrosis. Kidney Int. 2011; 80(9):915-25. Epub 20110803. doi: 10.1038/ki.2011.217. PubMed PMID: 21814171.
73. Sironi M, Martinez F O, D'Ambrosio D, Gattorno M, Polentarutti N, Locati M, Gregorio A, Iellem A, Cassatella M A, Van Damme J, Sozzani S, Martini A, Sinigaglia F, Vecchi A, Mantovani A. Differential regulation of chemokine production by Fcgamma receptor engagement in human monocytes: association of CCL1 with a distinct form of M2 monocyte activation (M2b, Type 2). J Leukoc Biol. 2006; 80(2):342-9. Epub 20060530. doi: 10.1189/jlb.1005586. PubMed PMID: 16735693.
74. Sundblad V, Morosi L G, Geffner J R, Rabinovich G A. Galectin-1: A Jack-of-All-Trades in the Resolution of Acute and Chronic Inflammation. J Immunol. 2017; 199(11):3721-30. doi: 10.4049/jimmunol.1701172. PubMed PMID: 29158348.
75. Toro-Dominguez D, Martorell-Marugan J, Goldman D, Petri M, Carmona-Saez P, Alarcon-Riquelme M E. Stratification of Systemic Lupus Erythematosus Patients Into Three Groups of Disease Activity Progression According to Longitudinal Gene Expression. Arthritis Rheumatol. 2018; 70(12):2025-35. Epub 20181027. doi: 10.1002/art.40653. PubMed PMID: 29938934; PMCID: PMC8326877.
76. Panousis N I, Bertsias G K, Ongen H, Gergianaki I, Tektonidou M G, Trachana M, Romano-Palumbo L, Bielser D, Howald C, Pamfil C, Fanouriakis A, Kosmara D, Repa A, Sidiropoulos P, Dermitzakis E T, Boumpas D T. Combined genetic and transcriptome analysis of patients with SLE: distinct, targetable signatures for susceptibility and severity. Ann Rheum Dis. 2019; 78(8):1079-89. Epub 20190605. doi: 10.1136/annrheumdis-2018-214379. PubMed PMID: 31167757; PMCID: PMC6691930.
77. Evrard M, Kwok I W H, Chong S Z, Teng K W W, Becht E, Chen J, Sieow J L, Penny H L, Ching G C, Devi S, Adrover J M, Li J L Y, Liong K H, Tan L, Poon Z, Foo S, Chua J W, Su I H, Balabanian K, Bachelerie F, Biswas S K, Larbi A, Hwang W Y K, Madan V, Koeffler H P, Wong S C, Newell E W, Hidalgo A, Ginhoux F, Ng L G. Developmental Analysis of Bone Marrow Neutrophils Reveals Populations Specialized in Expansion, Trafficking, and Effector Functions. Immunity. 2018; 48(2):364-79 e8. doi: 10.1016/j.immuni.2018.02.002. PubMed PMID: 29466759.
78. Mistry P, Nakabo S, O'Neil L, Goel R R, Jiang K, Carmona-Rivera C, Gupta S, Chan D W, Carlucci P M, Wang X, Naz F, Manna Z, Dey A, Mehta N N, Hasni S, Dell'Orso S, Gutierrez-Cruz G, Sun H W, Kaplan M J. Transcriptomic, epigenetic, and functional analyses implicate neutrophil diversity in the pathogenesis of systemic lupus erythematosus. Proc Natl Acad Sci USA. 2019; 116(50):25222-8. Epub 20191121. doi: 10.1073/pnas.1908576116. PubMed PMID: 31754025; PMCID: PMC6911190.
79. Pezzato E, Dona M, Sartor L, Dell'Aica I, Benelli R, Albini A, Garbisa S. Proteinase-3 directly activates MMP-2 and degrades gelatin and Matrigel; differential inhibition by (−)epigallocatechin-3-gallate. J Leukoc Biol. 2003; 74(1):88-94. doi: 10.1189/jlb.0203086. PubMed PMID: 12832446.
80. Davies M, Barrett A J, Travis J, Sanders E, Coles G A. The degradation of human glomerular basement membrane with purified lysosomal proteinases: evidence for the pathogenic role of the polymorphonuclear leucocyte in glomerulonephritis. Clin Sci Mol Med. 1978; 54(3):233-40. doi: 10.1042/cs0540233. PubMed PMID: 630800.
81. Turck J, Pollock A S, Lee L K, Marti H P, Lovett D H. Matrix metalloproteinase 2 (gelatinase A) regulates glomerular mesangial cell proliferation and differentiation. J Biol Chem. 1996; 271(25):15074-83. doi: 10.1074/jbc.271.25.15074. PubMed PMID: 8663054.
82. Toro-Dominguez D, Lopez-Dominguez R, Garcia Moreno A, Villatoro-Garcia J A, Martorell-Marugan J, Goldman D, Petri M, Wojdyla D, Pons-Estel B A, Isenberg D, Morales-Montes de Oca G, Trejo-Zambrano M I, Garcia Gonzalez B, Rosetti F, Gomez-Martin D, Romero-Diaz J, Carmona-Saez P, Alarcon-Riquelme M E. Differential Treatments Based on Drug-induced Gene Expression Signatures and Longitudinal Systemic Lupus Erythematosus Stratification. Sci Rep. 2019; 9(1):15502. Epub 20191029. doi: 10.1038/s41598-019-51616-9. PubMed PMID: 31664045; PMCID: PMC6820741.
83. Cruikshank W W, Greenstein J L, Theodore A C, Center D M. Lymphocyte chemoattractant factor induces CD4-dependent intracytoplasmic signaling in lymphocytes. J Immunol. 1991; 146(9):2928-34. PubMed PMID: 1673145.
84. Qi J C, Wang J, Mandadi S, Tanaka K, Roufogalis B D, Madigan M C, Lai K, Yan F, Chong B H, Stevens R L, Krilis S A. Human and mouse mast cells use the tetraspanin CD9 as an alternate interleukin-16 receptor. Blood. 2006; 107(1):135-42. Epub 20050906. doi: 10.1182/blood-2005-03-1312. PubMed PMID: 16144798; PMCID: PMC1895361.
85. Louache F, Debili N, Marandin A, Coulombel L, Vainchenker W. Expression of CD4 by human hematopoietic progenitors [see comments]. Blood. 1994; 84(10):3344-55. doi: 10.1182/blood.V84.10.3344.3344.
86. Biswas P, Mantelli B, Sica A, Malnati M, Panzeri C, Saccani A, Hasson H, Vecchi A, Saniabadi A, Lusso P, Lazzarin A, Beretta A. Expression of CD4 on human peripheral blood neutrophils. Blood. 2003; 101(11):4452-6. Epub 20030116. doi: 10.1182/blood-2002-10-3056. PubMed PMID: 12531788.
87. Lazareth H, Henique C, Lenoir O, Puelles V G, Flamant M, Bollee G, Fligny C, Camus M, Guyonnet L, Millien C, Gaillard F, Chipont A, Robin B, Fabrega S, Dhaun N, Camerer E, Kretz O, Grahammer F, Braun F, Huber T B, Nochy D, Mandet C, Bruneval P, Mesnard L, Thervet E, Karras A, Le Naour F, Rubinstein E, Boucheix C, Alexandrou A, Moeller M J, Bouzigues C, Tharaux P L. The tetraspanin CD9 controls migration and proliferation of parietal epithelial cells and glomerular disease progression. Nat Commun. 2019; 10(1):3303. Epub 20190724. doi: 10.1038/s41467-019-11013-2. PubMed PMID: 31341160; PMCID: PMC6656772.
88. Tao J, Wang H, Wang S X, Yu F, Zhao M H. The predictive value of crescents in the disease progression of lupus nephritis based on the 2018 International Society of Nephrology/Renal Pathology Society Revision System: a large cohort study from China. Ren Fail. 2020; 42(1):166-72. doi: 10.1080/0886022X.2020.1726385. PubMed PMID: 32054387; PMCID: PMC7034106.
89. Zhang W, Yuan M, Hong L, Zhou Q, Chen W, Yang S, Yang Q, Chen W, Yu X. Clinical outcomes of lupus nephritis patients with different proportions of crescents. Lupus. 2016; 25(14):1532-41. Epub 20160407. doi: 10.1177/0961203316642312. PubMed PMID: 27060063.
90. Carlucci P M, Purmalek M M, Dey A K, Temesgen-Oyelakin Y, Sakhardande S, Joshi A A, Lerman J B, Fike A, Davis M, Chung J H, Playford M P, Naqi M, Mistry P, Gutierrez-Cruz G, Dell'Orso S, Naz F, Salahuddin T, Natarajan B, Manna Z, Tsai W L, Gupta S, Grayson P, Teague H, Chen M Y, Sun H W, Hasni S, Mehta N N, Kaplan M J. Neutrophil subsets and their gene signature associate with vascular inflammation and coronary atherosclerosis in lupus. JCI Insight. 2018; 3(8). Epub 20180419. doi: 10.1172/jci.insight.99276. PubMed PMID: 29669944; PMCID: PMC5931124.
91. Zhang T, Li H, Vanarsa K, Gidley G, Mok C C, Petri M, Saxena R, Mohan C. Association of Urine sCD163 With Proliferative Lupus Nephritis, Fibrinoid Necrosis, Cellular Crescents and Intrarenal M2 Macrophages. Front Immunol. 2020; 11:671. Epub 20200415. doi: 10.3389/fimmu.2020.00671. PubMed PMID: 32351512; PMCID: PMC7174755.
92. Kiani A N, Johnson K, Chen C, Diehl E, Hu H, Vasudevan G, Singh S, Magder L S, Knechtle S J, Petri M. Urine osteoprotegerin and monocyte chemoattractant protein-1 in lupus nephritis. J Rheumatol. 2009; 36(10):2224-30. Epub 20090731. doi: 10.3899/jrheum.081112. PubMed PMID: 19648301.
93. Rubinstein T, Pitashny M, Levine B, Schwartz N, Schwartzman J, Weinstein E, Pego-Reigosa J M, Lu T Y, Isenberg D, Rahman A, Putterman C. Urinary neutrophil gelatinase-associated lipocalin as a novel biomarker for disease activity in lupus nephritis. Rheumatology (Oxford). 2010; 49(5):960-71. Epub 20100209. doi: 10.1093/rheumatology/kep468. PubMed PMID: 20144927; PMCID: PMC2853702.
94. Mejia-Vilet J M, Shapiro J P, Zhang X L, Cruz C, Zimmerman G, Mendez-Perez R A, Caño-Verduzco M L, Parikh S V, Nagaraja H N, Morales-Buenrostro L E, Rovin B H. Association Between Urinary Epidermal Growth Factor and Renal Prognosis in Lupus Nephritis. Arthritis Rheumatol. 2021; 73(2):244-54. Epub 20210112. doi: 10.1002/art.41507. PubMed PMID: 32892508.

What is claimed is:

1. A method of diagnosing lupus nephritis in a subject, the method comprising the steps of:
(a) obtaining a biological sample from a subject;
(b) detecting a presence of at least one biomarker in the sample, wherein at least one of the biomarkers is IL-16, Galectin-1, CD163, CD206, FOLR2, proteinase 3 (PRTN3), or a combination thereof; and
(c) diagnosing the subject as having lupus nephritis if at least one of IL-16, Galectin-1, CD163, CD 206, FOLR2, or PRTN3 is detected in the sample.

2. The method of claim 1, wherein the biomarkers IL-16, Galectin-1, CD163, CD206, FOLR2 and PRTN3 are detected in the sample from the subject.

3. The method of claim 1 or claim 2, wherein the method further comprises detecting the presence of at least one additional biomarker in the sample, wherein the biomarker is from Table 1.

4. The method of any of claims 1-3, wherein presence of at least one biomarker in the sample is displayed on an instrument.

5. The method of any of claims 1-4, wherein the method further comprises treating the subject diagnosed with lupus nephritis with at least one immunosuppressant, at least one corticosteroid, at least one B-lymphocyte stimulator specific inhibitor, rituximab, or any combination thereof.

6. The method of any of claims 1-5, wherein the sample is a whole blood, serum, plasma, or urine.

7. A method of predicting a subject's risk of developing lupus nephritis, the method comprising the steps of:
(a) obtaining a biological sample from a subject;
(b) determining a level of at least one biomarker in the sample, wherein at least one of the biomarkers is IL-16, Galectin-1, CD163, CD206, FOLR2, proteinase 3 (PRTN3), or a combination thereof;
(c) comparing the level of the at least one biomarker determined in step (b) with a control level for the same biomarker; and
(d) predicting whether the subject at risk of developing lupus nephritis based on the comparison in step (c).

8. The method of claim 7, wherein the level of IL-16, Galectin-1, CD163, CD206, FOLR2, and PRTN3 are determined in the sample from the subject.

9. The method of claim 7 or claim 8, wherein the method further comprises determining the level of at least one additional biomarker in the sample, wherein the biomarker is from Table 1.

10. The method of any of any of claims 7-9, wherein the method further comprises determining that the subject is at risk of developing lupus nephritis when the level of the at least one biomarker determined in step (b) is higher than the control level or that the subject is not at risk of developing lupus nephritis when the level of the at least one biomarker determined in step (b) is lower than the control level.

11. The method of claim 10, wherein the method further comprises treating the patient for lupus nephritis if the subject is determined to be at risk of developing lupus nephritis.

12. The method of claim 11, wherein the subject is treated with at least one immunosuppressant, at least on corticosteroid, a B-lymphocyte stimulator specific inhibitor, rituximab, or a combination thereof.

13. The method of claim 12, wherein the at least one immunosuppressant is cyclosporine, tacrolimus, a calcineurin-inhibitor, cyclophosphamide, hydroxychloroquine, azathioprine, mycophenolate, or any combination thereof.

14. The method of any of claim 12 or claim 13, wherein the corticosteroid is prednisone, prednisolone, methylprednisolone, or any combination thereof.

15. The method of any of claims 7-14, wherein concentration of at least one biomarker in the sample is displayed on an instrument.

16. The method of claims 7-15, wherein the sample is a whole blood, serum, plasma, or urine.

17. A method of determining whether a subject suffering from lupus nephritis is responding to treatment, the method comprising the steps of:
(a) obtaining a biological sample from a subject being treated for lupus nephritis;
(b) determining a level at least one biomarker in the sample, wherein at least one of the biomarkers are IL-16, Galectin-1, CD163, CD206, FOLR2, proteinase 3 (PRTN3), or a combination thereof;
(c) comparing the level of the at least one biomarker determined in step (b) with a control level for the same biomarker; and
(d) determining that the subject is responding to treatment for lupus nephritis if the level of the at least one biomarker determined in step (b) is less than the control level for the same biomarker or that the subject is not responding to treatment for lupus nephritis if the level of the at least one biomarker determine in step (b) is the same as or greater than the control level for the same biomarker.

18. The method of claim 17, wherein the control level for a biomarker is the level of the biomarker obtained from the subject prior to the start of treatment.

19. The method of claim 17 or claim 18, wherein the level of IL-16, Galectin-1, CD163, CD206, FOLR2, and PRTN3 are determined in the sample from the subject.

20. The method of claim 19, wherein the sample is obtained from the subject after 3 months of treatment.

21. The method of any of claims 17-20, wherein the method further comprises determining the level of at least one additional biomarker in the sample, wherein the biomarker is from Table 1.

22. The method of any of claims 17-21, wherein the method comprises monitoring the subject receiving treatment for the lupus nephritis.

23. The method of any of claims 17-22, wherein subject is treated with at least one immunosuppressant, at least on corticosteroid, a B-lymphocyte stimulator specific inhibitor, rituximab, or a combination thereof.

24. The method of claim 23, wherein the at least one immunosuppressant is cyclosporine, tacrolimus, a calcineurin-inhibitor, cyclophosphamide, hydroxychloroquine, azathioprine, mycophenolate, or any combination thereof.

25. The method of any of claim 23 or claim 24, wherein the corticosteroid is prednisone, prednisolone, methylprednisolone, or any combination thereof.

26. The method of any of claims 17-25, wherein the level of at least one biomarker in the sample is displayed on an instrument.

27. The method of claims 17-26, wherein the sample is a whole blood, serum, plasma, or urine.

28. The method of any of claims 1-27, wherein the lupus nephritis is proliferative lupus nephritis.

29. A method for determining a type or grade of lupus nephritis in a subject, the method comprising the steps of:
(a) obtaining a biological sample from a subject being treated for lupus nephritis;
(b) determining a level at least one biomarker in the sample, wherein at least one of the biomarkers are IL-16, CD163, Catalase, PRTN3, S100A8, Azurocidin, and MMP8, or a combination thereof; and
(c) determining that the type or grade of the lupus nephritis is proliferative when the level of the at least one biomarker determined in step (b) is higher than the control level for the same biomarker or that the type or grade of the lupus nephritis is pure membranous when the level of the at least one biomarker determined in step (b) is less than the control level for the same biomarker.

30. An article of manufacture comprising a set of reagents to measure the levels of a panel of biomarkers in a biological sample, wherein the panel of biomarkers comprises IL-16, Galectin-1, CD163, CD206, FOLR2, and proteinase 3 (PRTN3) and optionally, at least one biomarker in Table 1, and wherein the set of reagents are bound to a solid support and specifically binds to the biomarkers.

31. The article of manufacture of claim 30, wherein the reagents are specific binding partners.

32. The article of manufacture of claim 31, wherein the specific binding partner is an antibody or antigen-binding fragment thereof. a peptide or a fragment thereof, or a combination thereof.

33. The article of manufacture of any of claims 30-32, wherein the solid support is a biochip, a microtiter plate, a stick, a bead or any combination thereof.

34. A test kit comprising the set of reagents of claim 30.
